SEQUENCE LISTING (44 sequences)

SEQ ID NO: 1 - protein, 50 aa, Araneus diadematus
SGCDVLVQALLEVVSALVSILGSSSIGQINYGASAQYTQMVGQSVAQALA

SEQ ID NO: 2 - protein, 30 aa, Araneus diadematus
SGCDVLVQALLEVVSALVSILGSSSIGQIN

SEQ ID NO: 3 - protein, 21 aa, Araneus diadematus
SGCDVLVQALLEVVSALVSIL

SEQ ID NO: 4 - protein, 1154 aa, Artificial Sequence
  recombinant spider silk protein ADF3KaiLargeNRSH1
MHHHHHHHHHHSSGSSLEVLFQGPARAGSGQQGPGQQGPGQQGPGQQGPYGPGASAAAAAAGGYGPGSGQQGPSQQGPGQQGPGGQGPYGPGASAAAAAAGGYGPGSGQQGPGGQGPYGPGSSAAAAAAGGNGPGSGQQGAGQQGPGQQGPGASAAAAAAGGYGPGSGQQGPGQQGPGGQGPYGPGASAAAAAAGGYGPGSGQGPGQQGPGGQGPYGPGASAAAAAAGGYGPGSGQQGPGQQGPGQQGPGGQGPYGPGASAAAAAAGGYGPGYGQQGPGQQGPGGQGPYGPGASAASAASGGYGPGSGQQGPGQQGPGGQGPYGPGASAAAAAAGGYGPGSGQQGPGQQGPGQQGPGQQGPGGQGPYGPGASAAAAAAGGYGPGSGQQGPGQQGPGQQGPGQQGPGQQGPGQQGPGQQGPGQQGPGQQGPGGQGAYGPGASAAAGAAGGYGPGSGQQGPGQQGPGQQGPGQQGPGQQGPGQQGPGQQGPGQQGPYGPGASAAAAAAGGYGPGSGQQGPGQQGPGQQGPGGQGPYGPGAASAAVSVSRARAGSGQQGPGQQGPGQQGPGQQGPYGPGASAAAAAAGGYGPGSGQQGPSQQGPGQQGPGGQGPYGPGASAAAAAAGGYGPGSGQQGPGGQGPYGPGSSAAAAAAGGNGPGSGQQGAGQQGPGQQGPGASAAAAAAGGYGPGSGQQGPGQQGPGGQGPYGPGASAAAAAAGGYGPGSGQGPGQQGPGGQGPYGPGASAAAAAAGGYGPGSGQQGPGQQGPGQQGPGGQGPYGPGASAAAAAAGGYGPGYGQQGPGQQGPGGQGPYGPGASAASAASGGYGPGSGQQGPGQQGPGGQGPYGPGASAAAAAAGGYGPGSGQQGPGQQGPGQQGPGQQGPGGQGPYGPGASAAAAAAGGYGPGSGQQGPGQQGPGQQGPGQQGPGQQGPGQQGPGQQGPGQQGPGQQGPGGQGAYGPGASAAAGAAGGYGPGSGQQGPGQQGPGQQGPGQQGPGQQGPGQQGPGQQGPGQQGPYGPGASAAAAAAGGYGPGSGQQGPGQQGPGQQGPGGQGPYGPGAASAAVSVGGYGPQSSSVPVASAVASRLSSPAASSRVSSAVSSLVSSGPTKHAALSNTISSVVSQVSASNPGLSGCDVLVQALLEVVSALVSIL

SEQ ID NO: 5 - protein, 24 aa, Artificial Sequence
  His tag and start codon
MHHHHHHHHHHSSGSSLEVLFQGP

SEQ ID NO: 6 - protein, 597 aa, Artificial Sequence
  Met-PRT380
MGPGQQGPYGPGASAAAAAGQNGPGSGQQGPGQSAAAAAGQYGPGQQGPGQQGPGSSAAAAAGPGQYGPGQQGPSASAAAAAGPGSGQQGPGASAAAAAGQYGPGQQGPGQQGPGSSAAAAAGQYGSGPGQQGPYGSAAAAAGPGSGQYGQGPYGPGASAAAAAGPGQYGPGQQGPSASAAAAAGSGQQGPGQYGPYASAAAAAGQYGSGPGQQGPYGPGQSAAAAAGSGQQGPGQQGPYASAAAAAGPGQQGPYGPGSSAAAAAGQYGYGPGQQGPYGPGASAAAAAGQNGPGSGQYGPGQQGPGQSAAAAAGPGQQGPYGPGASAAAAAGQYGPGQQGPGQYGPGSSAAAAAGPGQQGPYGPGSSAAAAAGQYGPGQQGPYGPGQSAAAAAGQYQQGPGQQGPYGPGASAAAAAGPGQQGPYGPGASAAAAAGPGQYGPGQQGPSASAAAAAGQYGSGPGQYGPYGPGQSAAAAAGPGSGQQGQGPYGPGASAAAAAGQYGPGQQGPYGPGQSAAAAAGPGSGQYGPGASAAAAAGQNGPGSGQYGPGQQGPGQSAAAAAGQYQQGPGQQGPYGPGASAAAAAGPGSGQQGPGAS

SEQ ID NO: 7 - protein, 590 aa, Artificial Sequence
  Met-PRT410
MGPGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGAS

SEQ ID NO: 8 - protein, 565 aa, Artificial Sequence
  Met-PRT468
MGPGQQGPYGPGASAAAAAAAGSNGPGSGQQGPGQSGQYGPGQQGPGQQGPGSSAAAAAAAGPGQYGPGQQGPSASAAAAAAAGPGSGQQGPGASGQYGPGQQGPGQQGPGSSAAAAAAAGSYGSGPGQQGPYGSAAAAAAAGPGSGQYGQGPYGPGASGPGQYGPGQQGPSASAAAAAAAGSGQQGPGQYGPYASAAAAAAAGSYGSGPGQQGPYGPGQSGSGQQGPGQQGPYASAAAAAAAGPGQQGPYGPGSSAAAAAAAGSYGYGPGQQGPYGPGASGQNGPGSGQYGPGQQGPGPSAAAAAAAGPGQQGPYGPGASAAAAAAAGSYGPGQQGPGQYGPGSSGPGQQGPYGPGSSAAAAAAAGSYGPGQQGPYGPGPSAAAAAAAGSYQQGPGQQGPYGPGASGPGQQGPYGPGASAAAAAAAGPGQYGPGQQGPSASAAAAAAAGSYGSGPGQYGPYGPGQSGPGSGQQGQGPYGPGASAAAAAAAGSYGPGQQGPYGPGPSAAAAAAAGPGSGQYGPGASGQNGPGSGQYGPGQQGPGPSAAAAAAAGPGSGQQGPGAS

SEQ ID NO: 9 - protein, 2364 aa, Artificial Sequence
  Met-PRT799
MGPGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGASGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGASGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGASGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGSSVDKLAAALEHHHHHH

SEQ ID NO: 10 - protein, 597 aa, Artificial Sequence
  Met-PRT313
MGPGGQGPYGPGASAAAAAGGNGPGSGQQGPGGSAAAAAGGYGPGGQGPGQQGPGSSAAAAAGPGGYGPGGQGPSASAAAAAGPGSGQQGPGASAAAAAGGYGPGGQGPGQQGPGSSAAAAAGGYGSGPGQQGPYGSAAAAAGPGSGGYGQGPYGPGASAAAAAGPGGYGPGGQGPSASAAAAAGSGQQGPGGYGPYASAAAAAGGYGSGPGQQGPYGPGGSAAAAAGSGQQGPGQQGPYASAAAAAGPGGQGPYGPGSSAAAAAGGYGYGPGGQGPYGPGASAAAAAGGNGPGSGGYGPGQQGPGGSAAAAAGPGGQGPYGPGASAAAAAGGYGPGGQGPGGYGPGSSAAAAAGPGGQGPYGPGSSAAAAAGGYGPGQQGPYGPGGSAAAAAGGYQQGPGGQGPYGPGASAAAAAGPGGQGPYGPGASAAAAAGPGGYGPGGQGPSASAAAAAGGYGSGPGGYGPYGPGGSAAAAAGPGSGQQGQGPYGPGASAAAAAGGYGPGQQGPYGPGGSAAAAAGPGSGGYGPGASAAAAAGGNGPGSGGYGPGQQGPGGSAAAAAGGYQQGPGGQGPYGPGASAAAAAGPGSGQQGPGAS

SEQ ID NO: 11 - protein, 601 aa, Artificial Sequence
  PRT410
MHHHHHHSSGSSGPGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGAS

SEQ ID NO: 12 - protein, 12 aa, Artificial Sequence
  HisTag
MHHHHHHSSGSS

SEQ ID NO: 13 - protein, 608 aa, Artificial Sequence
  PRT380
MHHHHHHSSGSSGPGQQGPYGPGASAAAAAGQNGPGSGQQGPGQSAAAAAGQYGPGQQGPGQQGPGSSAAAAAGPGQYGPGQQGPSASAAAAAGPGSGQQGPGASAAAAAGQYGPGQQGPGQQGPGSSAAAAAGQYGSGPGQQGPYGSAAAAAGPGSGQYGQGPYGPGASAAAAAGPGQYGPGQQGPSASAAAAAGSGQQGPGQYGPYASAAAAAGQYGSGPGQQGPYGPGQSAAAAAGSGQQGPGQQGPYASAAAAAGPGQQGPYGPGSSAAAAAGQYGYGPGQQGPYGPGASAAAAAGQNGPGSGQYGPGQQGPGQSAAAAAGPGQQGPYGPGASAAAAAGQYGPGQQGPGQYGPGSSAAAAAGPGQQGPYGPGSSAAAAAGQYGPGQQGPYGPGQSAAAAAGQYQQGPGQQGPYGPGASAAAAAGPGQQGPYGPGASAAAAAGPGQYGPGQQGPSASAAAAAGQYGSGPGQYGPYGPGQSAAAAAGPGSGQQGQGPYGPGASAAAAAGQYGPGQQGPYGPGQSAAAAAGPGSGQYGPGASAAAAAGQNGPGSGQYGPGQQGPGQSAAAAAGQYQQGPGQQGPYGPGASAAAAAGPGSGQQGPGAS

SEQ ID NO: 14 - protein, 576 aa, Artificial Sequence
  PRT468
MHHHHHHSSGSSGPGQQGPYGPGASAAAAAAAGSNGPGSGQQGPGQSGQYGPGQQGPGQQGPGSSAAAAAAAGPGQYGPGQQGPSASAAAAAAAGPGSGQQGPGASGQYGPGQQGPGQQGPGSSAAAAAAAGSYGSGPGQQGPYGSAAAAAAAGPGSGQYGQGPYGPGASGPGQYGPGQQGPSASAAAAAAAGSGQQGPGQYGPYASAAAAAAAGSYGSGPGQQGPYGPGQSGSGQQGPGQQGPYASAAAAAAAGPGQQGPYGPGSSAAAAAAAGSYGYGPGQQGPYGPGASGQNGPGSGQYGPGQQGPGPSAAAAAAAGPGQQGPYGPGASAAAAAAAGSYGPGQQGPGQYGPGSSGPGQQGPYGPGSSAAAAAAAGSYGPGQQGPYGPGPSAAAAAAAGSYQQGPGQQGPYGPGASGPGQQGPYGPGASAAAAAAAGPGQYGPGQQGPSASAAAAAAAGSYGSGPGQYGPYGPGQSGPGSGQQGQGPYGPGASAAAAAAAGSYGPGQQGPYGPGPSAAAAAAAGPGSGQYGPGASGQNGPGSGQYGPGQQGPGPSAAAAAAAGPGSGQQGPGAS

SEQ ID NO: 15 - protein, 2375 aa, Artificial Sequence
  PRT799
MHHHHHHSSGSSGPGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGASGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGASGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGASGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGSSVDKLAAALEHHHHHH

SEQ ID NO: 16 - protein, 608 aa, Artificial Sequence
  PRT313
MHHHHHHSSGSSGPGGQGPYGPGASAAAAAGGNGPGSGQQGPGGSAAAAAGGYGPGGQGPGQQGPGSSAAAAAGPGGYGPGGQGPSASAAAAAGPGSGQQGPGASAAAAAGGYGPGGQGPGQQGPGSSAAAAAGGYGSGPGQQGPYGSAAAAAGPGSGGYGQGPYGPGASAAAAAGPGGYGPGGQGPSASAAAAAGSGQQGPGGYGPYASAAAAAGGYGSGPGQQGPYGPGGSAAAAAGSGQQGPGQQGPYASAAAAAGPGGQGPYGPGSSAAAAAGGYGYGPGGQGPYGPGASAAAAAGGNGPGSGGYGPGQQGPGGSAAAAAGPGGQGPYGPGASAAAAAGGYGPGGQGPGGYGPGSSAAAAAGPGGQGPYGPGSSAAAAAGGYGPGQQGPYGPGGSAAAAAGGYQQGPGGQGPYGPGASAAAAAGPGGQGPYGPGASAAAAAGPGGYGPGGQGPSASAAAAAGGYGSGPGGYGPYGPGGSAAAAAGPGSGQQGQGPYGPGASAAAAAGGYGPGQQGPYGPGGSAAAAAGPGSGGYGPGASAAAAAGGNGPGSGGYGPGQQGPGGSAAAAAGGYQQGPGGQGPYGPGASAAAAAGPGSGQQGPGAS

SEQ ID NO: 17 - protein, 601 aa, Artificial Sequence
  PRT399
MHHHHHHSSGSSGPGGQGPYGPGASAAAAAGGNGPGSGQQGPGGSGGYGPGGQGPGQQGPGSSAAAAAGPGGYGPGGQGPSASAAAAAGPGSGQQGPGASGGYGPGGQGPGQQGPGSSAAAAAGGYGSGPGQQGPYGSAAAAAGPGSGGYGQGPYGPGASGPGGYGPGGQGPSASAAAAAGSGQQGPGGYGPYASAAAAAGGYGSGPGQQGPYGPGGSGSGQQGPGQQGPYASAAAAAGPGGQGPYGPGSSAAAAAGGYGYGPGGQGPYGPGASGGNGPGSGGYGPGQQGPGGSAAAAAGPGGQGPYGPGASAAAAAGGYGPGGQGPGGYGPGSSGPGGQGPYGPGSSAAAAAGGYGPGQQGPYGPGGSAAAAAGGYQQGPGGQGPYGPGASGPGGQGPYGPGASAAAAAGPGGYGPGGQGPSASAAAAAGGYGSGPGGYGPYGPGGSGPGSGQQGQGPYGPGASAAAAAGGYGPGQQGPYGPGGSAAAAAGPGSGGYGPGASGGNGPGSGGYGPGQQGPGGSAAAAAGGYQQGPGGQGPYGPGASAAAAAGGYGSGPGQQGPYGPGGSGSGQQGPGQQGPYASAAAAAGPGSGQQGPGAS

SEQ ID NO: 18 - protein, 590 aa, Artificial Sequence
  Met-PRT399
MGPGGQGPYGPGASAAAAAGGNGPGSGQQGPGGSGGYGPGGQGPGQQGPGSSAAAAAGPGGYGPGGQGPSASAAAAAGPGSGQQGPGASGGYGPGGQGPGQQGPGSSAAAAAGGYGSGPGQQGPYGSAAAAAGPGSGGYGQGPYGPGASGPGGYGPGGQGPSASAAAAAGSGQQGPGGYGPYASAAAAAGGYGSGPGQQGPYGPGGSGSGQQGPGQQGPYASAAAAAGPGGQGPYGPGSSAAAAAGGYGYGPGGQGPYGPGASGGNGPGSGGYGPGQQGPGGSAAAAAGPGGQGPYGPGASAAAAAGGYGPGGQGPGGYGPGSSGPGGQGPYGPGSSAAAAAGGYGPGQQGPYGPGGSAAAAAGGYQQGPGGQGPYGPGASGPGGQGPYGPGASAAAAAGPGGYGPGGQGPSASAAAAAGGYGSGPGGYGPYGPGGSGPGSGQQGQGPYGPGASAAAAAGGYGPGQQGPYGPGGSAAAAAGPGSGGYGPGASGGNGPGSGGYGPGQQGPGGSAAAAAGGYQQGPGGQGPYGPGASAAAAAGGYGSGPGQQGPYGPGGSGSGQQGPGQQGPYASAAAAAGPGSGQQGPGAS

SEQ ID NO: 19 - protein, 612 aa, Artificial Sequence
  Met-PRT720
MGPGQQGPYGPGASAAAAAGQNGPGSGQQGPGQSGQYGPGQQGPGQQGPGSSAAAAAGPGQYVLIGPGQQVLIGPSASAAAAAGPGSGQQGPGASGQYGPGQQGPGQQGPGSSAAAAAGSYGSVLIGPGQQVLIGPYGSAAAAAGPGSGQYGQGPYGPGASGPGQYGPGQQGPSASAAAAAGSGQQVLIGPGQYVLIGPYASAAAAAGQYGSGPGQQGPYGPGQSGSGQQGPGQQGPYASAAAAAGPGQQVLIGPYVLIGPGSSAAAAAGQYGYGPGQQGPYGPGASGQNGPGSGQYGPGQQGPGQSAAAAAGPGQQVLIGPYVLIGPGASAAAAAGQYGPGQQGPGQYGPGSSGPGQQGPYGPGSSAAAAAGSYGPGQQVLIGPYVLIGPGPSAAAAAGQYQQGPGQQGPYGPGASGPGQQGPYGPGASAAAAAGPGQYVLIGPGQQVLIGPSASAAAAAGQYGSGPGQYGPYGPGQSGPGSGQQGQGPYGPGASAAAAAGSYGPGQQVLIGPYVLIGPGPSAAAAAGPGSGQYGPGASGQNGPGSGQYGPGQQGPGQSAAAAAGQYQQVLIGPGQQGPYVLIGPGASAAAAAGPGSGQQVLIGPGASVLI

SEQ ID NO: 20 - protein, 592 aa, Artificial Sequence
  Met-PRT665
MGPGQQGPYGPGASAAAAAAAGSNGPGSGQQGPGQSGQYGPGQQGPGQQGPGSSAAAAAAAGPGQYVLIGPGQQGPSASAAAAAAAGPGSGQQGPGASGQYGPGQQGPGQQGPGSSAAAAAAAGSYGSVLIGPGQQGPYGSAAAAAAAGPGSGQYGQGPYGPGASGPGQYGPGQQGPSASAAAAAAAGSGQQVLIGPGQYGPYASAAAAAAAGSYGSGPGQQGPYGPGQSGSGQQGPGQQGPYASAAAAAAAGPGQQVLIGPYGPGSSAAAAAAAGSYGYGPGQQGPYGPGASGQNGPGSGQYGPGQQGPGPSAAAAAAAGPGQQVLIGPYGPGASAAAAAAAGSYGPGQQGPGQYGPGSSGPGQQGPYGPGSSAAAAAAAGSYGPGQQVLIGPYGPGPSAAAAAAAGSYQQGPGQQGPYGPGASGPGQQGPYGPGASAAAAAAAGPGQYVLIGPGQQGPSASAAAAAAAGSYGSGPGQYGPYGPGQSGPGSGQQGQGPYGPGASAAAAAAAGSYGPGQQVLIGPYGPGPSAAAAAAAGPGSGQYGPGASGQNGPGSGQYGPGQQGPGPSAAAAAAAGPGSGQQGPGASVLI

SEQ ID NO: 21 - protein, 619 aa, Artificial Sequence
  Met-PRT666
MGPGQQGPYGPGASAAAAAAAGSNGPGSGQQGPGQSGQYGPGQQGPGQQGPGSSAAAAAAAGPGQYVLIGPGQQVLIGPSASAAAAAAAGPGSGQQGPGASGQYGPGQQGPGQQGPGSSAAAAAAAGSYGSVLIGPGQQVLIGPYGSAAAAAAAGPGSGQYGQGPYGPGASGPGQYGPGQQGPSASAAAAAAAGSGQQVLIGPGQYVLIGPYASAAAAAAAGSYGSGPGQQGPYGPGQSGSGQQGPGQQGPYASAAAAAAAGPGQQVLIGPYVLIGPGSSAAAAAAAGSYGYGPGQQGPYGPGASGQNGPGSGQYGPGQQGPGPSAAAAAAAGPGQQVLIGPYVLIGPGASAAAAAAAGSYGPGQQGPGQYGPGSSGPGQQGPYGPGSSAAAAAAAGSYGPGQQVLIGPYVLIGPGPSAAAAAAAGSYQQGPGQQGPYGPGASGPGQQGPYGPGASAAAAAAAGPGQYVLIGPGQQVLIGPSASAAAAAAAGSYGSGPGQYGPYGPGQSGPGSGQQGQGPYGPGASAAAAAAAGSYGPGQQVLIGPYVLIGPGPSAAAAAAAGPGSGQYGPGASGQNGPGSGQYGPGQQGPGPSAAAAAAAGPGSGQQVLIGPGASVLI

SEQ ID NO: 22 - protein, 590 aa, Artificial Sequence; identical to SEQ ID NO: 7
  Met-PRT410

SEQ ID NO: 23 - protein, 565 aa, Artificial Sequence; identical to SEQ ID NO: 8
  Met-PRT468

SEQ ID NO: 24 - protein, 623 aa, Artificial Sequence
  PRT468
MHHHHHHSSGSSGPGQQGPYGPGASAAAAAGQNGPGSGQQGPGQSGQYGPGQQGPGQQGPGSSAAAAAGPGQYVLIGPGQQVLIGPSASAAAAAGPGSGQQGPGASGQYGPGQQGPGQQGPGSSAAAAAGSYGSVLIGPGQQVLIGPYGSAAAAAGPGSGQYGQGPYGPGASGPGQYGPGQQGPSASAAAAAGSGQQVLIGPGQYVLIGPYASAAAAAGQYGSGPGQQGPYGPGQSGSGQQGPGQQGPYASAAAAAGPGQQVLIGPYVLIGPGSSAAAAAGQYGYGPGQQGPYGPGASGQNGPGSGQYGPGQQGPGQSAAAAAGPGQQVLIGPYVLIGPGASAAAAAGQYGPGQQGPGQYGPGSSGPGQQGPYGPGSSAAAAAGSYGPGQQVLIGPYVLIGPGPSAAAAAGQYQQGPGQQGPYGPGASGPGQQGPYGPGASAAAAAGPGQYVLIGPGQQVLIGPSASAAAAAGQYGSGPGQYGPYGPGQSGPGSGQQGQGPYGPGASAAAAAGSYGPGQQVLIGPYVLIGPGPSAAAAAGPGSGQYGPGASGQNGPGSGQYGPGQQGPGQSAAAAAGQYQQVLIGPGQQGPYVLIGPGASAAAAAGPGSGQQVLIGPGASVLI

SEQ ID NO: 25 - protein, 603 aa, Artificial Sequence
  PRT665
MHHHHHHSSGSSGPGQQGPYGPGASAAAAAAAGSNGPGSGQQGPGQSGQYGPGQQGPGQQGPGSSAAAAAAAGPGQYVLIGPGQQGPSASAAAAAAAGPGSGQQGPGASGQYGPGQQGPGQQGPGSSAAAAAAAGSYGSVLIGPGQQGPYGSAAAAAAAGPGSGQYGQGPYGPGASGPGQYGPGQQGPSASAAAAAAAGSGQQVLIGPGQYGPYASAAAAAAAGSYGSGPGQQGPYGPGQSGSGQQGPGQQGPYASAAAAAAAGPGQQVLIGPYGPGSSAAAAAAAGSYGYGPGQQGPYGPGASGQNGPGSGQYGPGQQGPGPSAAAAAAAGPGQQVLIGPYGPGASAAAAAAAGSYGPGQQGPGQYGPGSSGPGQQGPYGPGSSAAAAAAAGSYGPGQQVLIGPYGPGPSAAAAAAAGSYQQGPGQQGPYGPGASGPGQQGPYGPGASAAAAAAAGPGQYVLIGPGQQGPSASAAAAAAAGSYGSGPGQYGPYGPGQSGPGSGQQGQGPYGPGASAAAAAAAGSYGPGQQVLIGPYGPGPSAAAAAAAGPGSGQYGPGASGQNGPGSGQYGPGQQGPGPSAAAAAAAGPGSGQQGPGASVLI

SEQ ID NO: 26 - protein, 630 aa, Artificial Sequence
  PRT666
MHHHHHHSSGSSGPGQQGPYGPGASAAAAAAAGSNGPGSGQQGPGQSGQYGPGQQGPGQQGPGSSAAAAAAAGPGQYVLIGPGQQVLIGPSASAAAAAAAGPGSGQQGPGASGQYGPGQQGPGQQGPGSSAAAAAAAGSYGSVLIGPGQQVLIGPYGSAAAAAAAGPGSGQYGQGPYGPGASGPGQYGPGQQGPSASAAAAAAAGSGQQVLIGPGQYVLIGPYASAAAAAAAGSYGSGPGQQGPYGPGQSGSGQQGPGQQGPYASAAAAAAAGPGQQVLIGPYVLIGPGSSAAAAAAAGSYGYGPGQQGPYGPGASGQNGPGSGQYGPGQQGPGPSAAAAAAAGPGQQVLIGPYVLIGPGASAAAAAAAGSYGPGQQGPGQYGPGSSGPGQQGPYGPGSSAAAAAAAGSYGPGQQVLIGPYVLIGPGPSAAAAAAAGSYQQGPGQQGPYGPGASGPGQQGPYGPGASAAAAAAAGPGQYVLIGPGQQVLIGPSASAAAAAAAGSYGSGPGQYGPYGPGQSGPGSGQQGQGPYGPGASAAAAAAAGSYGPGQQVLIGPYVLIGPGPSAAAAAAAGPGSGQYGPGASGQNGPGSGQYGPGQQGPGPSAAAAAAAGPGSGQQVLIGPGASVLI

SEQ ID NO: 27 - protein, 593 aa, Artificial Sequence
  M_PRT888
MGSSGPGVLGPYGPGASAAAAAGQNGPGSGVLGPGQSGQYGPGVLGPGVLGPGSSAAAAAGPGQYGPGVLGPSASAAAAAGPGSGVLGPGASGQYGPGVLGPGVLGPGSSAAAAAGQYGSGPGVLGPYGSAAAAAGPGSGQYGQGPYGPGASGPGQYGPGVLGPSASAAAAAGSGVLGPGQYGPYASAAAAAGQYGSGPGVLGPYGPGQSGSGVLGPGVLGPYASAAAAAGPGVLGPYGPGSSAAAAAGQYGYGPGVLGPYGPGASGQNGPGSGQYGPGVLGPGQSAAAAAGPGVLGPYGPGASAAAAAGQYGPGVLGPGQYGPGSSGPGVLGPYGPGSSAAAAAGQYGPGVLGPYGPGQSAAAAAGQYVLGPGVLGPYGPGASGPGVLGPYGPGASAAAAAGPGQYGPGVLGPSASAAAAAGQYGSGPGQYGPYGPGQSGPGSGVLGQGPYGPGASAAAAAGQYGPGVLGPYGPGQSAAAAAGPGSGQYGPGASGQNGPGSGQYGPGVLGPGQSAAAAAGQYVLGPGVLGPYGPGASAAAAAGQYGSGPGVLGPYGPGQSGSGVLGPGVLGPYASAAAAAGPGSGVLGPGAS

SEQ ID NO: 28 - protein, 590 aa, Artificial Sequence
  M_PRT965
MGPGTSGPYGPGASAAAAAGANGPGSGTSGPGASGAYGPGTSGPGTSGPGSSAAAAAGPGAYGPGTSGPSASAAAAAGPGSGTSGPGASGAYGPGTSGPGTSGPGSSAAAAAGAYGSGPGTSGPYGSAAAAAGPGSGAYGAGPYGPGASGPGAYGPGTSGPSASAAAAAGSGTSGPGAYGPYASAAAAAGAYGSGPGTSGPYGPGASGSGTSGPGTSGPYASAAAAAGPGTSGPYGPGSSAAAAAGAYGYGPGTSGPYGPGASGANGPGSGAYGPGTSGPGASAAAAAGPGTSGPYGPGASAAAAAGAYGPGTSGPGAYGPGSSGPGTSGPYGPGSSAAAAAGAYGPGTSGPYGPGASAAAAAGAYTSGPGTSGPYGPGASGPGTSGPYGPGASAAAAAGPGAYGPGTSGPSASAAAAAGAYGSGPGAYGPYGPGASGPGSGTSGAGPYGPGASAAAAAGAYGPGTSGPYGPGASAAAAAGPGSGAYGPGASGANGPGSGAYGPGTSGPGASAAAAAGAYTSGPGTSGPYGPGASAAAAAGAYGSGPGTSGPYGPGASGSGTSGPGTSGPYASAAAAAGPGSGTSGPGAS

SEQ ID NO: 29 - protein, 593 aa, Artificial Sequence
  M_PRT889
MGSSGPGVLGPYGPGASAAAAAGINGPGSGVLGPGISGIYGPGVLGPGVLGPGSSAAAAAGPGIYGPGVLGPSASAAAAAGPGSGVLGPGASGIYGPGVLGPGVLGPGSSAAAAAGIYGSGPGVLGPYGSAAAAAGPGSGIYGIGPYGPGASGPGIYGPGVLGPSASAAAAAGSGVLGPGIYGPYASAAAAAGIYGSGPGVLGPYGPGISGSGVLGPGVLGPYASAAAAAGPGVLGPYGPGSSAAAAAGIYGYGPGVLGPYGPGASGINGPGSGIYGPGVLGPGISAAAAAGPGVLGPYGPGASAAAAAGIYGPGVLGPGIYGPGSSGPGVLGPYGPGSSAAAAAGIYGPGVLGPYGPGISAAAAAGIYVLGPGVLGPYGPGASGPGVLGPYGPGASAAAAAGPGIYGPGVLGPSASAAAAAGIYGSGPGIYGPYGPGISGPGSGVLGIGPYGPGASAAAAAGIYGPGVLGPYGPGISAAAAAGPGSGIYGPGASGINGPGSGIYGPGVLGPGISAAAAAGIYVLGPGVLGPYGPGASAAAAAGIYGSGPGVLGPYGPGISGSGVLGPGVLGPYASAAAAAGPGSGVLGPGAS

SEQ ID NO: 30 - protein, 590 aa, Artificial Sequence
  M_PRT916
MGPGVIGPYGPGASAAAAAGLNGPGSGVIGPGLSGLYGPGVIGPGVIGPGSSAAAAAGPGLYGPGVIGPSASAAAAAGPGSGVIGPGASGLYGPGVIGPGVIGPGSSAAAAAGLYGSGPGVIGPYGSAAAAAGPGSGLYGLGPYGPGASGPGLYGPGVIGPSASAAAAAGSGVIGPGLYGPYASAAAAAGLYGSGPGVIGPYGPGLSGSGVIGPGVIGPYASAAAAAGPGVIGPYGPGSSAAAAAGLYGYGPGVIGPYGPGASGLNGPGSGLYGPGVIGPGLSAAAAAGPGVIGPYGPGASAAAAAGLYGPGVIGPGLYGPGSSGPGVIGPYGPGSSAAAAAGLYGPGVIGPYGPGLSAAAAAGLYVIGPGVIGPYGPGASGPGVIGPYGPGASAAAAAGPGLYGPGVIGPSASAAAAAGLYGSGPGLYGPYGPGLSGPGSGVIGLGPYGPGASAAAAAGLYGPGVIGPYGPGLSAAAAAGPGSGLYGPGASGLNGPGSGLYGPGVIGPGLSAAAAAGLYVIGPGVIGPYGPGASAAAAAGLYGSGPGVIGPYGPGLSGSGVIGPGVIGPYASAAAAAGPGSGVIGPGAS

SEQ ID NO: 31 - protein, 590 aa, Artificial Sequence
  M_ PRT918
MGPGVFGPYGPGASAAAAAGINGPGSGVFGPGISGIYGPGVFGPGVFGPGSSAAAAAGPGIYGPGVFGPSASAAAAAGPGSGVFGPGASGIYGPGVFGPGVFGPGSSAAAAAGIYGSGPGVFGPYGSAAAAAGPGSGIYGIGPYGPGASGPGIYGPGVFGPSASAAAAAGSGVFGPGIYGPYASAAAAAGIYGSGPGVFGPYGPGISGSGVFGPGVFGPYASAAAAAGPGVFGPYGPGSSAAAAAGIYGYGPGVFGPYGPGASGINGPGSGIYGPGVFGPGISAAAAAGPGVFGPYGPGASAAAAAGIYGPGVFGPGIYGPGSSGPGVFGPYGPGSSAAAAAGIYGPGVFGPYGPGISAAAAAGIYVFGPGVFGPYGPGASGPGVFGPYGPGASAAAAAGPGIYGPGVFGPSASAAAAAGIYGSGPGIYGPYGPGISGPGSGVFGIGPYGPGASAAAAAGIYGPGVFGPYGPGISAAAAAGPGSGIYGPGASGINGPGSGIYGPGVFGPGISAAAAAGIYVFGPGVFGPYGPGASAAAAAGIYGSGPGVFGPYGPGISGSGVFGPGVFGPYASAAAAAGPGSGVFGPGAS

SEQ ID NO: 32 - protein, 565 aa, Artificial Sequence
  M_PRT699
MGPGVLGPYGPGASAAAAAAAGSNGPGSGVLGPGQSGQYGPGVLGPGVLGPGSSAAAAAAAGPGQYGPGVLGPSASAAAAAAAGPGSGVLGPGASGQYGPGVLGPGVLGPGSSAAAAAAAGSYGSGPGVLGPYGSAAAAAAAGPGSGQYGQGPYGPGASGPGQYGPGVLGPSASAAAAAAAGSGVLGPGQYGPYASAAAAAAAGSYGSGPGVLGPYGPGQSGSGVLGPGVLGPYASAAAAAAAGPGVLGPYGPGSSAAAAAAAGSYGYGPGVLGPYGPGASGQNGPGSGQYGPGVLGPGPSAAAAAAAGPGVLGPYGPGASAAAAAAAGSYGPGVLGPGQYGPGSSGPGVLGPYGPGSSAAAAAAAGSYGPGVLGPYGPGPSAAAAAAAGSYVLGPGVLGPYGPGASGPGVLGPYGPGASAAAAAAAGPGQYGPGVLGPSASAAAAAAAGSYGSGPGQYGPYGPGQSGPGSGVLGQGPYGPGASAAAAAAAGSYGPGVLGPYGPGPSAAAAAAAGPGSGQYGPGASGQNGPGSGQYGPGVLGPGPSAAAAAAAGPGSGVLGPGAS

SEQ ID NO: 33 - protein, 565 aa, Artificial Sequence
  M_PRT698
MGPGVLGPYGPGASAAAAAAAGSNGPGSGVLGPGISGIYGPGVLGPGVLGPGSSAAAAAAAGPGIYGPGVLGPSASAAAAAAAGPGSGVLGPGASGIYGPGVLGPGVLGPGSSAAAAAAAGSYGSGPGVLGPYGSAAAAAAAGPGSGIYGIGPYGPGASGPGIYGPGVLGPSASAAAAAAAGSGVLGPGIYGPYASAAAAAAAGSYGSGPGVLGPYGPGISGSGVLGPGVLGPYASAAAAAAAGPGVLGPYGPGSSAAAAAAAGSYGYGPGVLGPYGPGASGINGPGSGIYGPGVLGPGPSAAAAAAAGPGVLGPYGPGASAAAAAAAGSYGPGVLGPGIYGPGSSGPGVLGPYGPGSSAAAAAAAGSYGPGVLGPYGPGPSAAAAAAAGSYVLGPGVLGPYGPGASGPGVLGPYGPGASAAAAAAAGPGIYGPGVLGPSASAAAAAAAGSYGSGPGIYGPYGPGISGPGSGVLGIGPYGPGASAAAAAAAGSYGPGVLGPYGPGPSAAAAAAAGPGSGIYGPGASGINGPGSGIYGPGVLGPGPSAAAAAAAGPGSGVLGPGAS

SEQ ID NO: 34 - protein, 565 aa, Artificial Sequence; identical to SEQ ID NO: 8
  M_PRT525

SEQ ID NO: 35 - protein, 601 aa, Artificial Sequence
  PRT888
MHHHHHHSSGSSGPGVLGPYGPGASAAAAAGQNGPGSGVLGPGQSGQYGPGVLGPGVLGPGSSAAAAAGPGQYGPGVLGPSASAAAAAGPGSGVLGPGASGQYGPGVLGPGVLGPGSSAAAAAGQYGSGPGVLGPYGSAAAAAGPGSGQYGQGPYGPGASGPGQYGPGVLGPSASAAAAAGSGVLGPGQYGPYASAAAAAGQYGSGPGVLGPYGPGQSGSGVLGPGVLGPYASAAAAAGPGVLGPYGPGSSAAAAAGQYGYGPGVLGPYGPGASGQNGPGSGQYGPGVLGPGQSAAAAAGPGVLGPYGPGASAAAAAGQYGPGVLGPGQYGPGSSGPGVLGPYGPGSSAAAAAGQYGPGVLGPYGPGQSAAAAAGQYVLGPGVLGPYGPGASGPGVLGPYGPGASAAAAAGPGQYGPGVLGPSASAAAAAGQYGSGPGQYGPYGPGQSGPGSGVLGQGPYGPGASAAAAAGQYGPGVLGPYGPGQSAAAAAGPGSGQYGPGASGQNGPGSGQYGPGVLGPGQSAAAAAGQYVLGPGVLGPYGPGASAAAAAGQYGSGPGVLGPYGPGQSGSGVLGPGVLGPYASAAAAAGPGSGVLGPGAS

SEQ ID NO: 36 - protein, 601 aa, Artificial Sequence
  PRT965
MHHHHHHSSGSSGPGTSGPYGPGASAAAAAGANGPGSGTSGPGASGAYGPGTSGPGTSGPGSSAAAAAGPGAYGPGTSGPSASAAAAAGPGSGTSGPGASGAYGPGTSGPGTSGPGSSAAAAAGAYGSGPGTSGPYGSAAAAAGPGSGAYGAGPYGPGASGPGAYGPGTSGPSASAAAAAGSGTSGPGAYGPYASAAAAAGAYGSGPGTSGPYGPGASGSGTSGPGTSGPYASAAAAAGPGTSGPYGPGSSAAAAAGAYGYGPGTSGPYGPGASGANGPGSGAYGPGTSGPGASAAAAAGPGTSGPYGPGASAAAAAGAYGPGTSGPGAYGPGSSGPGTSGPYGPGSSAAAAAGAYGPGTSGPYGPGASAAAAAGAYTSGPGTSGPYGPGASGPGTSGPYGPGASAAAAAGPGAYGPGTSGPSASAAAAAGAYGSGPGAYGPYGPGASGPGSGTSGAGPYGPGASAAAAAGAYGPGTSGPYGPGASAAAAAGPGSGAYGPGASGANGPGSGAYGPGTSGPGASAAAAAGAYTSGPGTSGPYGPGASAAAAAGAYGSGPGTSGPYGPGASGSGTSGPGTSGPYASAAAAAGPGSGTSGPGAS

SEQ ID NO: 37 - protein, 601 aa, Artificial Sequence
  PRT889
MHHHHHHSSGSSGPGVLGPYGPGASAAAAAGINGPGSGVLGPGISGIYGPGVLGPGVLGPGSSAAAAAGPGIYGPGVLGPSASAAAAAGPGSGVLGPGASGIYGPGVLGPGVLGPGSSAAAAAGIYGSGPGVLGPYGSAAAAAGPGSGIYGIGPYGPGASGPGIYGPGVLGPSASAAAAAGSGVLGPGIYGPYASAAAAAGIYGSGPGVLGPYGPGISGSGVLGPGVLGPYASAAAAAGPGVLGPYGPGSSAAAAAGIYGYGPGVLGPYGPGASGINGPGSGIYGPGVLGPGISAAAAAGPGVLGPYGPGASAAAAAGIYGPGVLGPGIYGPGSSGPGVLGPYGPGSSAAAAAGIYGPGVLGPYGPGISAAAAAGIYVLGPGVLGPYGPGASGPGVLGPYGPGASAAAAAGPGIYGPGVLGPSASAAAAAGIYGSGPGIYGPYGPGISGPGSGVLGIGPYGPGASAAAAAGIYGPGVLGPYGPGISAAAAAGPGSGIYGPGASGINGPGSGIYGPGVLGPGISAAAAAGIYVLGPGVLGPYGPGASAAAAAGIYGSGPGVLGPYGPGISGSGVLGPGVLGPYASAAAAAGPGSGVLGPGAS

SEQ ID NO: 38 - protein, 601 aa, Artificial Sequence
  PRT916
MHHHHHHSSGSSGPGVIGPYGPGASAAAAAGLNGPGSGVIGPGLSGLYGPGVIGPGVIGPGSSAAAAAGPGLYGPGVIGPSASAAAAAGPGSGVIGPGASGLYGPGVIGPGVIGPGSSAAAAAGLYGSGPGVIGPYGSAAAAAGPGSGLYGLGPYGPGASGPGLYGPGVIGPSASAAAAAGSGVIGPGLYGPYASAAAAAGLYGSGPGVIGPYGPGLSGSGVIGPGVIGPYASAAAAAGPGVIGPYGPGSSAAAAAGLYGYGPGVIGPYGPGASGLNGPGSGLYGPGVIGPGLSAAAAAGPGVIGPYGPGASAAAAAGLYGPGVIGPGLYGPGSSGPGVIGPYGPGSSAAAAAGLYGPGVIGPYGPGLSAAAAAGLYVIGPGVIGPYGPGASGPGVIGPYGPGASAAAAAGPGLYGPGVIGPSASAAAAAGLYGSGPGLYGPYGPGLSGPGSGVIGLGPYGPGASAAAAAGLYGPGVIGPYGPGLSAAAAAGPGSGLYGPGASGLNGPGSGLYGPGVIGPGLSAAAAAGLYVIGPGVIGPYGPGASAAAAAGLYGSGPGVIGPYGPGLSGSGVIGPGVIGPYASAAAAAGPGSGVIGPGAS

SEQ ID NO: 39 - protein, 601 aa, Artificial Sequence
  PRT918
MHHHHHHSSGSSGPGVFGPYGPGASAAAAAGINGPGSGVFGPGISGIYGPGVFGPGVFGPGSSAAAAAGPGIYGPGVFGPSASAAAAAGPGSGVFGPGASGIYGPGVFGPGVFGPGSSAAAAAGIYGSGPGVFGPYGSAAAAAGPGSGIYGIGPYGPGASGPGIYGPGVFGPSASAAAAAGSGVFGPGIYGPYASAAAAAGIYGSGPGVFGPYGPGISGSGVFGPGVFGPYASAAAAAGPGVFGPYGPGSSAAAAAGIYGYGPGVFGPYGPGASGINGPGSGIYGPGVFGPGISAAAAAGPGVFGPYGPGASAAAAAGIYGPGVFGPGIYGPGSSGPGVFGPYGPGSSAAAAAGIYGPGVFGPYGPGISAAAAAGIYVFGPGVFGPYGPGASGPGVFGPYGPGASAAAAAGPGIYGPGVFGPSASAAAAAGIYGSGPGIYGPYGPGISGPGSGVFGIGPYGPGASAAAAAGIYGPGVFGPYGPGISAAAAAGPGSGIYGPGASGINGPGSGIYGPGVFGPGISAAAAAGIYVFGPGVFGPYGPGASAAAAAGIYGSGPGVFGPYGPGISGSGVFGPGVFGPYASAAAAAGPGSGVFGPGAS

SEQ ID NO: 40 - protein, 576 aa, Artificial Sequence
  PRT699
MHHHHHHSSGSSGPGVLGPYGPGASAAAAAAAGSNGPGSGVLGPGQSGQYGPGVLGPGVLGPGSSAAAAAAAGPGQYGPGVLGPSASAAAAAAAGPGSGVLGPGASGQYGPGVLGPGVLGPGSSAAAAAAAGSYGSGPGVLGPYGSAAAAAAAGPGSGQYGQGPYGPGASGPGQYGPGVLGPSASAAAAAAAGSGVLGPGQYGPYASAAAAAAAGSYGSGPGVLGPYGPGQSGSGVLGPGVLGPYASAAAAAAAGPGVLGPYGPGSSAAAAAAAGSYGYGPGVLGPYGPGASGQNGPGSGQYGPGVLGPGPSAAAAAAAGPGVLGPYGPGASAAAAAAAGSYGPGVLGPGQYGPGSSGPGVLGPYGPGSSAAAAAAAGSYGPGVLGPYGPGPSAAAAAAAGSYVLGPGVLGPYGPGASGPGVLGPYGPGASAAAAAAAGPGQYGPGVLGPSASAAAAAAAGSYGSGPGQYGPYGPGQSGPGSGVLGQGPYGPGASAAAAAAAGSYGPGVLGPYGPGPSAAAAAAAGPGSGQYGPGASGQNGPGSGQYGPGVLGPGPSAAAAAAAGPGSGVLGPGAS

SEQ ID NO: 41 - protein, 576 aa, Artificial Sequence
  PRT698
MHHHHHHSSGSSGPGVLGPYGPGASAAAAAAAGSNGPGSGVLGPGISGIYGPGVLGPGVLGPGSSAAAAAAAGPGIYGPGVLGPSASAAAAAAAGPGSGVLGPGASGIYGPGVLGPGVLGPGSSAAAAAAAGSYGSGPGVLGPYGSAAAAAAAGPGSGIYGIGPYGPGASGPGIYGPGVLGPSASAAAAAAAGSGVLGPGIYGPYASAAAAAAAGSYGSGPGVLGPYGPGISGSGVLGPGVLGPYASAAAAAAAGPGVLGPYGPGSSAAAAAAAGSYGYGPGVLGPYGPGASGINGPGSGIYGPGVLGPGPSAAAAAAAGPGVLGPYGPGASAAAAAAAGSYGPGVLGPGIYGPGSSGPGVLGPYGPGSSAAAAAAAGSYGPGVLGPYGPGPSAAAAAAAGSYVLGPGVLGPYGPGASGPGVLGPYGPGASAAAAAAAGPGIYGPGVLGPSASAAAAAAAGSYGSGPGIYGPYGPGISGPGSGVLGIGPYGPGASAAAAAAAGSYGPGVLGPYGPGPSAAAAAAAGPGSGIYGPGASGINGPGSGIYGPGVLGPGPSAAAAAAAGPGSGVLGPGAS

SEQ ID NO: 42 - protein, 17 aa, Artificial Sequence
  Hinge+His-tag
SSVDKLAAALEHHHHHH

SEQ ID NO: 43 - protein, 1179 aa, Artificial Sequence
  Met-PRT966
MGPGVFGPYGPGASAAAAAGINGPGSGVFGPGISGIYGPGVFGPGVFGPGSSAAAAAGPGIYGPGVFGPSASAAAAAGPGSGVFGPGASGIYGPGVFGPGVFGPGSSAAAAAGIYGSGPGVFGPYGSAAAAAGPGSGIYGIGPYGPGASGPGIYGPGVFGPSASAAAAAGSGVFGPGIYGPYASAAAAAGIYGSGPGVFGPYGPGISGSGVFGPGVFGPYASAAAAAGPGVFGPYGPGSSAAAAAGIYGYGPGVFGPYGPGASGINGPGSGIYGPGVFGPGISAAAAAGPGVFGPYGPGASAAAAAGIYGPGVFGPGIYGPGSSGPGVFGPYGPGSSAAAAAGIYGPGVFGPYGPGISAAAAAGIYVFGPGVFGPYGPGASGPGVFGPYGPGASAAAAAGPGIYGPGVFGPSASAAAAAGIYGSGPGIYGPYGPGISGPGSGVFGIGPYGPGASAAAAAGIYGPGVFGPYGPGISAAAAAGPGSGIYGPGASGINGPGSGIYGPGVFGPGISAAAAAGIYVFGPGVFGPYGPGASAAAAAGIYGSGPGVFGPYGPGISGSGVFGPGVFGPYASAAAAAGPGSGVFGPGASGPGVFGPYGPGASAAAAAGINGPGSGVFGPGISGIYGPGVFGPGVFGPGSSAAAAAGPGIYGPGVFGPSASAAAAAGPGSGVFGPGASGIYGPGVFGPGVFGPGSSAAAAAGIYGSGPGVFGPYGSAAAAAGPGSGIYGIGPYGPGASGPGIYGPGVFGPSASAAAAAGSGVFGPGIYGPYASAAAAAGIYGSGPGVFGPYGPGISGSGVFGPGVFGPYASAAAAAGPGVFGPYGPGSSAAAAAGIYGYGPGVFGPYGPGASGINGPGSGIYGPGVFGPGISAAAAAGPGVFGPYGPGASAAAAAGIYGPGVFGPGIYGPGSSGPGVFGPYGPGSSAAAAAGIYGPGVFGPYGPGISAAAAAGIYVFGPGVFGPYGPGASGPGVFGPYGPGASAAAAAGPGIYGPGVFGPSASAAAAAGIYGSGPGIYGPYGPGISGPGSGVFGIGPYGPGASAAAAAGIYGPGVFGPYGPGISAAAAAGPGSGIYGPGASGINGPGSGIYGPGVFGPGISAAAAAGIYVFGPGVFGPYGPGASAAAAAGIYGSGPGVFGPYGPGISGSGVFGPGVFGPYASAAAAAGPGSGVFGPGAS

SEQ ID NO: 44 - protein, 1190 aa, Artificial Sequence
  PRT966
MHHHHHHSSGSSGPGVFGPYGPGASAAAAAGINGPGSGVFGPGISGIYGPGVFGPGVFGPGSSAAAAAGPGIYGPGVFGPSASAAAAAGPGSGVFGPGASGIYGPGVFGPGVFGPGSSAAAAAGIYGSGPGVFGPYGSAAAAAGPGSGIYGIGPYGPGASGPGIYGPGVFGPSASAAAAAGSGVFGPGIYGPYASAAAAAGIYGSGPGVFGPYGPGISGSGVFGPGVFGPYASAAAAAGPGVFGPYGPGSSAAAAAGIYGYGPGVFGPYGPGASGINGPGSGIYGPGVFGPGISAAAAAGPGVFGPYGPGASAAAAAGIYGPGVFGPGIYGPGSSGPGVFGPYGPGSSAAAAAGIYGPGVFGPYGPGISAAAAAGIYVFGPGVFGPYGPGASGPGVFGPYGPGASAAAAAGPGIYGPGVFGPSASAAAAAGIYGSGPGIYGPYGPGISGPGSGVFGIGPYGPGASAAAAAGIYGPGVFGPYGPGISAAAAAGPGSGIYGPGASGINGPGSGIYGPGVFGPGISAAAAAGIYVFGPGVFGPYGPGASAAAAAGIYGSGPGVFGPYGPGISGSGVFGPGVFGPYASAAAAAGPGSGVFGPGASGPGVFGPYGPGASAAAAAGINGPGSGVFGPGISGIYGPGVFGPGVFGPGSSAAAAAGPGIYGPGVFGPSASAAAAAGPGSGVFGPGASGIYGPGVFGPGVFGPGSSAAAAAGIYGSGPGVFGPYGSAAAAAGPGSGIYGIGPYGPGASGPGIYGPGVFGPSASAAAAAGSGVFGPGIYGPYASAAAAAGIYGSGPGVFGPYGPGISGSGVFGPGVFGPYASAAAAAGPGVFGPYGPGSSAAAAAGIYGYGPGVFGPYGPGASGINGPGSGIYGPGVFGPGISAAAAAGPGVFGPYGPGASAAAAAGIYGPGVFGPGIYGPGSSGPGVFGPYGPGSSAAAAAGIYGPGVFGPYGPGISAAAAAGIYVFGPGVFGPYGPGASGPGVFGPYGPGASAAAAAGPGIYGPGVFGPSASAAAAAGIYGSGPGIYGPYGPGISGPGSGVFGIGPYGPGASAAAAAGIYGPGVFGPYGPGISAAAAAGPGSGIYGPGASGINGPGSGIYGPGVFGPGISAAAAAGIYVFGPGVFGPYGPGASAAAAAGIYGSGPGVFGPYGPGISGSGVFGPGVFGPYASAAAAAGPGSGVFGPGAS

(19) United States
(12) Patent Application Publication
(10) Pub. No.: US 2021/0040648 A1
(43) Pub. Date: Feb. 11, 2021

(54) Method for Manufacturing Protein Fiber

(71) Applicant: Spiber Inc., Tsuruoka-shi, Yamagata (JP)

(72) Inventors: Yunosuke Abe, Tsuruoka-shi, Yamagata (JP); Hirotada Ando, Kariya-shi, Aichi (JP)

(73) Assignee: Spiber Inc., Tsuruoka-shi, Yamagata (JP)

(21) Appl. No.: 16/965,626

(22) PCT Filed: Jan. 31, 2019
(86) PCT No.: PCT/JP2019/003463

§ 371 Date: Jul. 29, 2020

(30) Foreign Application Priority Data
Jan. 31, 2018 (JP) 2018-014710

(51) Int. Cl. D01F 4/02; D01D 5/06

(52) U.S. Cl. CPC D01F 4/02; D01D 5/06

(57) ABSTRACT

The present invention relates to a method for manufacturing a protein fiber, the method including a step of bringing a spinning raw liquid containing a spider silk protein and a solvent into contact with a coagulation liquid to coagulate the spider silk protein, in which the coagulation liquid contains a ketone.

### TECHNICAL FIELD

The present invention relates to a method for manufacturing a protein fiber.

### BACKGROUND ART

As a method for manufacturing artificial fibers, a wet-type spinning method and a dry-wet-type spinning method in which a spinning raw liquid discharged from a nozzle is coagulated with a coagulation bath solution to form fibers are conventionally known. The wet-type spinning method and the dry-wet-type spinning method are also used when manufacturing protein fibers containing protein as a main component (refer to, for example, Patent Literature 1).

As a method for manufacturing protein fibers by the wet-type spinning method and the dry-wet-type spinning method, the following procedure is known: using a protein solution obtained by dissolving protein in a solvent as a dope solution (spinning raw liquid), the dope solution is extruded from a spinneret to a coagulation liquid in a desolvation bath, the solvent is desorbed from the dope solution and fibers are formed to obtain undrawn yarn, and thereby protein fibers are obtained (refer to, for example, Patent Literature 2).

Known solvents for dissolving proteins include dimethyl sulfoxide, N,N-dimethylformamide, N,N-dimethylacetamide, formic acid, and the like. In addition, in manufacture of protein fibers by the wet-type spinning method and the dry-wet-type spinning method, lower alcohols such as methanol, ethanol, and 2-propanol are generally used as a coagulation liquid for removing the solvent and forming fibers. For example, Non-Patent Literature 1 and Non-Patent Literature 2 disclose that regenerated silk fibroin was dissolved in formic acid and introduced into a coagulation liquid of methanol, and thereby favorable coagulation and fiber-forming ability were obtained.

### CITATION LIST

### Patent Literature

[Patent Literature 1] Japanese Patent No. 5540154
[Patent Literature 2] International Publication No.

2013/065651

### Non-Patent Literature

[Non-Patent Literature 1] Int. J. Biol. Macromol., Vol. 41, 2007, pp. 168-172
[Non-Patent Literature 2] Int. J. Biol. Macromol., Vol. 34, 2004, pp. 89-105

### SUMMARY OF INVENTION

### Problems to be Solved by the Invention

When protein fibers are manufactured by the wet-type spinning method and the dry-wet-type spinning method using a structural protein of high molecular weight, which is a material likely to have a high utility value in the future, such as silk fibroin and spider silk fibroin, coagulation liquids for forming protein fibers are very limited. Among them, lower alcohols such as methanol, ethanol, and 2-propanol are widely used as a coagulation liquid for structural proteins because they can be obtained relatively inexpensively and have a fiber-forming ability. However, it can be said that the coagulation liquid for structural proteins has not been sufficiently studied so far.

The present invention has been made in view of the above problems of the related art, and an object of the present invention is to provide a method for manufacturing a protein fiber, the method imparting an excellent fiber-forming ability (yarn-making properties) to spider silk proteins.

### Means for Solving the Problems

The inventors of the present invention have found that the above-mentioned object can be achieved by combining a spinning raw liquid containing a spider silk protein with a coagulation liquid containing a ketone. The present invention is based on this novel finding.

The present invention relates to each of the following inventions, for example.

[1] A method for manufacturing a protein fiber, the method including:

a step of bringing a spinning raw liquid containing a spider silk protein and a solvent into contact with a coagulation liquid to coagulate the spider silk protein,

in which the coagulation liquid contains a ketone.

[2] The manufacturing method according to [1], in which the ketone is a water-soluble ketone.

[3] The manufacturing method according to [1] or [2], in which the ketone is acetone.

[4] The manufacturing method according to any one of [1] to [3], in which a content of the ketone in the coagulation liquid is 60% by mass or more with respect to a total amount of the coagulation liquid.

[5] The manufacturing method according to any one of [1] to [4], in which the coagulation liquid further contains at least one selected from the group consisting of the solvent, a dissolution promoter, and water.

[6] The manufacturing method according to any one of [1] to [5], in which the solvent contains formic acid.

[7] The manufacturing method according to any one of [1] to [6],

in which the coagulation liquid further contains formic acid, and

a content of the formic acid is 40% by mass or less with a total content of the ketone and the formic acid being 100% by mass.

[8] The manufacturing method according to any one of [1] to [7], in which the spinning raw liquid further contains a dissolution promoter.

[9] The manufacturing method according to any one of [1] to [8], in which the spider silk protein is a hydrophilic spider silk protein.

[10] The manufacturing method according to [9], in which a content of the ketone in the coagulation liquid is 70% by mass or more with respect to a total amount of the coagulation liquid.

[11] The manufacturing method according to any one of [1] to [10], further including a step of drawing the coagulated spider silk protein.

### Effects of the Invention

A method for manufacturing a protein fiber of the present invention imparts an excellent fiber-forming ability (yarn-making properties) to spider silk proteins. In addition to being able to secure the fiber-forming ability (yarn-making properties), the method for manufacturing a protein fiber of the present invention can adopt higher speed yarn-making conditions because a maximum draw ratio drawable between a nozzle and a roller is increased, as compared with a conventional methanol coagulation liquid. The method for manufacturing a protein fiber of the present invention can provide thinner protein fibers.

### BRIEF DESCRIPTION OF DRAWINGS

FIG. 1 is a schematic diagram showing an example of a domain sequence of a spider fibroin.

FIG. 2 is a schematic diagram showing an example of a domain sequence of a spider fibroin.

FIG. 3 is a schematic diagram showing an example of a domain sequence of a spider fibroin.

FIG. 4 is an explanatory view schematically showing an example of a spinning apparatus for manufacturing protein fibers.

FIG. 5 shows photographs showing an appearance of protein fibers manufactured in Manufacture Example 1.

FIG. 6 shows photographs showing an appearance of protein fibers manufactured in Manufacture Example 2.

### EMBODIMENTS FOR CARRYING OUT THE INVENTION

Hereinafter, embodiments for carrying out the present invention will be described in detail with reference to the drawings in some cases, but the present invention is not limited to the following embodiments.

[Method for Manufacturing Protein Fiber]

A method for manufacturing a protein fiber according to the present embodiment includes a step of bringing a spinning raw liquid containing a spider silk protein and a solvent into contact with a coagulation liquid to coagulate the spider silk protein (spinning step). The coagulation liquid contains a ketone. The method for manufacturing a protein fiber of the present embodiment can be carried out according to known spinning methods such as wet-type spinning and dry-wet-type spinning

<Spinning Raw Liquid>

The spinning raw liquid (dope solution) used in the manufacturing method of the present invention contains the spider silk protein and the solvent.

(Spider Silk Protein)

The spider silk protein that is a raw material is not particularly limited, but it may be a protein manufactured from microorganisms or the like by genetic recombination technology, may be a protein manufactured synthetically, or may be a naturally-occurring protein.

The spider silk protein of the present embodiment includes a naturally-occurring spider silk protein and a modified spider silk protein (hereinafter, also referred to as “modified fibroin”). In the present specification, the term “naturally-occurring spider silk protein” means a spider silk protein having the same amino acid sequence as that of a naturally-occurring spider silk protein (spider fibroin and the like), and the term “modified spider silk protein” or “modified fibroin” means a spider silk protein having an amino acid sequence that differs from that of a naturally-occurring spider silk protein.

Examples of naturally-occurring spider silk proteins include spider fibroins produced by spiders such as large spinneret dragline silk proteins, weft thread silk proteins, and minor ampullate gland silk proteins. Large spinneret dragline silk has both high stress and stretchability because it has a repeated region consisting of a crystalline region and a non-crystalline region (also referred to as an amorphous region). Weft thread spider silk has a characteristic of not having a crystalline region but and having a repeated region consisting of a non-crystalline region. The weft thread is inferior in stress as compared with the large spinneret dragline silk, but has high stretchability.

The large spinneret dragline silk protein is characterized by having excellent toughness because it is produced from the major ampullate gland. Examples of large spinneret dragline silk proteins include major ampullate spidroins MaSp1 and MaSp2 derived from Nephila clavipes, and ADF3 and ADF4 derived from Araneus diadematus. ADF3 is one of the two major dragline silk proteins of Araneus diadematus. A spider silk protein may be a spider silk protein derived from these dragline silk proteins. Spider silk proteins derived from ADF3 have excellent characteristics of being relatively easily synthesized and having excellent tensile strength and toughness.

A weft thread protein is produced in the flagelliform gland of spiders. Examples of weft thread proteins include a flagelliform silk protein derived from Nephila clavipes.

Examples of spider fibroins produced by spiders further include spider silk proteins produced by spiders belonging to the genus Araneus such as Araneus ventricosus, Araneus diadematus, Araneus pinguis, Araneus pentagrammicus, and Araneus nojimai, spiders belonging to the genus Neoscona such as Neoscona scylla, Neoscona nautica, Neoscona adianta, and Neoscona scylloides, spiders belonging to the genus Pronus such as Pronous minutes, spiders belonging to the genus Cyrtarachne such as Cyrtarachne bufo and Cyrtarachne inaequalis, spiders belonging to the genus Gasteracantha such as Gasteracantha kuhli and Gasteracantha mammosa, spiders belonging to the genus Ordgarius such as Ordgarius hobsoni and Ordgarius sexspinosus, spiders belonging to the genus Argiope such as Argiope amoena, Argiope minuta, and Argiope bruennich, spiders belonging to the genus Arachnura such as Arachnura logio, spiders belonging to the genus Acusilas such as Acusilas coccineus, spiders belonging to the genus Cytophora such as Cyrtophora moluccensis, Cyrtophora exanthematica, and Cyrtophora unicolor, spiders belonging to the genus Poltys such as Poltys illepidus, spiders belonging to the genus Cyclosa such as Cyclosa octotuberculata, Cyclosa sedeculata, Cyclosa vallata, and Cyclosa atrata, and spiders belonging to the genus Chorizopes such as Chorizopes nipponicus; and spider silk proteins produced by spiders belonging to the genus Tetragnatha such as Tetragnatha praedonia, Tetragnatha maxillosa, Tetragnatha extensa, and Tetragnatha squamata, spiders belonging to the genus Leucauge such as Leucauge magnifica, Leucauge blanda, and Leucauge subblanda, spiders belonging to the genus Nephila such as Nephila clavata and Nephila pilipes, spiders belonging to the genus Menosira such as Menosira ornata, spiders belonging to the genus Dyschiriognatha such as Dyschiriognatha tenera, spiders belonging to the genus Latrodectus such as Latrodectus mactans, Latrodectus hasseltii, Latrodectus geometricus, and Latrodectus tredecimguttatus, and spiders belonging to the family Tetragnathidae such as spiders belonging to the genus Euprosthenops.

More specific examples of spider silk proteins produced by spiders include fibroin-3 (adf-3) [derived from Araneus diadematus] (GenBank Accession Number AAC47010 (amino acid sequence), U47855 (base sequence)), fibroin-4 (adf-4) [derived from Araneus diadematus] (GenBank Accession Number AAC47011 (amino acid sequence), U47856 (base sequence)), dragline silk protein spidroin 1 [derived from Nephila clavipes] (GenBank Accession Number AAC04504 (amino acid sequence), U37520 (base sequence)), major ampullate spidroin 1 [derived from Latrodectus hesperus] (GenBank Accession Number ABR68856 (amino acid sequence), EF595246 (base sequence)), dragline silk protein spidroin 2 [derived from Nephila clavata] (GenBank Accession Number AAL32472 (amino acid sequence), AF441245 (base sequence)), major ampullate spidroin 1 [derived from Euprosthenops australis] (GenBank Accession Number CAJ00428 (amino acid sequence), AJ973155 (base sequence)) and major ampullate spidroin 2 [Euprosthenops australis] (GenBank Accession Number CAM32249.1 (amino acid sequence), AM490169 (base sequence)), minor ampullate silk protein 1 [Nephila clavipes] (GenBank Accession Number AAC14589.1 (amino acid sequence)), minor ampullate silk protein 2 [Nephila clavipes] (GenBank Accession Number AAC14591.1 (amino acid sequence)), minor ampullate spidroin-like protein [Nephilengys cruentata] (GenBank Accession Number ABR37278.1 (amino acid sequence)), and the like.

The spider silk protein of the present embodiment may be, for example, a protein having a domain sequence represented by Formula 1: [(A)ₙ motif-REP]ₘ or Formula 2: [(A)ₙ motif-REP]ₘ-(A)ₙ motif. The spider silk protein of the present embodiment may further have amino acid sequences (N-terminal sequence and C-terminal sequence) added to either or both of the N-terminal side and the C-terminal side of the domain sequence. The N-terminal sequence and the C-terminal sequence are typically regions not having repetitions of amino acid motifs characteristic of fibroin, and consist of amino acids of about 100 residues, but they are not limited thereto.

The term “domain sequence” in the present specification refers to an amino acid sequence which produces a crystalline region (which typically corresponds to (A)ₙ motif of an amino acid sequence) and a non-crystalline region (which typically corresponds to REP of an amino acid sequence), which are particular to fibroin, and means an amino acid sequence represented by Formula 1: [(A)ₙ motif-REP]ₘ or Formula 2: [(A)ₙ motif-REP]ₘ-(A)ₙ motif. The (A)ₙ motif indicates an amino acid sequence mainly including alanine residues, and the number of amino acid residues is 2 to 27. The number of amino acid residues of the (A)ₙ motif may be 2 to 20, 4 to 27, 4 to 20, 8 to 20, 10 to 20, 4 to 16, 8 to 16, or 10 to 16. In addition, it is sufficient that a ratio of the number of alanine residues be 40% or more with respect to a total number of amino acid residues in the (A)ₙ motif, and it may be 60% or more, 70% or more, 80% or more, 83% or more, 85% or more, 86% or more, 90% or more, 95% or more, or 100% (which means that the (A)ₙ motif consists of only alanine residues). At least seven of a plurality of (A)ₙ motifs present in the domain sequence may consist of only alanine residues. REP represents an amino acid sequence consisting of 2 to 200 amino acid residues. REP may be an amino acid sequence consisting of 10 to 200 amino acid residues. m indicates an integer of 2 to 300, and it may be an integer of 10 to 300. The plurality of (A)ₙ motifs may be the same amino acid sequences or different amino acid sequences. A plurality of REP's may be the same amino acid sequences or different amino acid sequences.

The modified spider silk protein (modified fibroin) may be a fibroin in which an amino acid sequence has been modified based on amino acid sequences of naturally-occurring spider fibroins (for example, a fibroin in which an amino acid sequence has been modified by modifying a genetic sequence of a cloned naturally-occurring spider fibroin), or a fibroin artificially designed and synthesized independently of naturally-occurring spider fibroins (for example, a fibroin having a desired amino acid sequence by chemically synthesizing nucleic acids encoding a designed amino acid sequence).

A modified fibroin can be obtained by, for example, performing modifications on an amino acid sequence such that it matches an amino acid sequence in which substitution, deletion, insertion, and/or addition of one or a plurality of amino acid residues has been carried out in a genetic sequence of a cloned naturally-occurring spider fibroin. Substitution, deletion, insertion, and/or addition of amino acid residues can be carried out by methods well known to those skilled in the art, such as site-directed mutagenesis. Specifically, they can be carried out according to a method described in documents such as Nucleic Acid Res. 10, 6487 (1982), and Methods in Enzymology, 100, 448 (1983).

Specific examples of modified fibroins include a modified fibroin derived from a large spinneret dragline silk protein produced from the major ampullate gland (first modified fibroin), a modified fibroin in which a content of glycine residues has been reduced (second modified fibroin), a modified fibroin in which a content of (A)ₙ motifs has been reduced (third modified fibroin), a modified fibroin in which a content of glycine residues and a content of (A)ₙ motifs have been reduced (fourth modified fibroin), a modified fibroin having a domain sequence that includes a region locally having a large hydrophobicity index (fifth modified fibroin), and a modified fibroin having a domain sequence in which a content of glutamine residues has been reduced (sixth modified fibroin).

Examples of the modified fibroin (first modified fibroin) derived from a large spinneret dragline silk protein produced from the major ampullate gland of spiders include a protein including a domain sequence represented by Formula 1: [(A)ₙ motif-REP]ₘ. Regarding the first modified fibroin, in Formula 1, n is preferably an integer of 3 to 20, more preferably an integer of 4 to 20, even more preferably an integer of 8 to 20, and still more preferably an integer of 10 to 20, further more preferably an integer of 4 to 16, particularly preferably an integer of 8 to 16, and most preferably an integer of 10 to 16. Regarding the first modified fibroin, in Formula 1, the number of amino acid residues constituting the REP is preferably 10 to 200 residues, more preferably 10 to 150 residues, even more preferably 20 to 100 residues, and still more preferably 20 to 75 residues. Regarding the first modified fibroin, a total number of residues of glycine residues, serine residues, and alanine residues contained in the amino acid sequence represented by Formula 1: [(A)ₙ motif-REP]ₘ is preferably 40% or more, more preferably 60% or more, and even more preferably 70% or more, with respect to a total number of amino acid residues.

The first modified fibroin includes a unit of the amino acid sequence represented by Formula 1: [(A)ₙ motif-REP]ₘ, and may be a protein in which an amino acid sequence shows a homology of 90% or more with an amino acid sequence having a C-terminal sequence set forth in any of SEQ ID NOs: 1 to 3, or an amino acid sequence set forth in any of SEQ ID NOs: 1 to 3.

The amino acid sequence set forth in SEQ ID NO: 1 is identical to an amino acid sequence consisting of 50 amino acid residues at the C-terminus of an amino acid sequence of ADF3 (GI: 1263287, NCBI); the amino acid sequence set forth in SEQ ID NO: 2 is identical to an amino acid sequence obtained by removing 20 residues from the C-terminus of the amino acid sequence set forth in SEQ ID NO: 1; and the amino acid sequence set forth in SEQ ID NO: 3 is identical to an amino acid sequence obtained by removing 29 residues from the C-terminus of the amino acid sequence set forth in SEQ ID NO: 1.

More specific examples of the first modified fibroin include a modified fibroin (1-i) including an amino acid sequence set forth in SEQ ID NO: 4, or a modified fibroin (1-ii) including an amino acid sequence having 90% or more sequence identity with the amino acid sequence set forth in SEQ ID NO: 4. It is preferable that the sequence identity be 95% or more.

The amino acid sequence set forth in SEQ ID NO: 4 is an amino acid sequence in which the first to thirteenth repeat regions are increased to approximately double, and which is mutated so that the translation is terminated at the 1154th amino acid residue, in an amino acid sequence of ADF3 in which an amino acid sequence (SEQ ID NO: 5) consisting of an initiation codon, His10 tag, and HRV3C protease (Human rhinovirus 3C protease) recognition site are added to the N-terminus. An amino acid sequence at the C-terminus of the amino acid sequence set forth in SEQ ID NO: 4 is identical to the amino acid sequence set forth in SEQ ID NO: 3.

The modified fibroin (1-i) may consist of the amino acid sequence set forth in SEQ ID NO: 4.

In the modified fibroin in which a content of glycine residues has been reduced (second modified fibroin), a domain sequence thereof has an amino acid sequence in which a content of glycine residues has been reduced, as compared to naturally-occurring spider fibroins. It can be said that the second modified fibroin has an amino acid sequence corresponding to an amino acid sequence in which one or a plurality of glycine residues in at least REP is substituted with another amino acid residue, as compared to naturally-occurring spider fibroins.

The second modified fibroin may be a modified fibroin in which a domain sequence thereof has, in at least one motif sequence selected from GGX and GPGXX in REP (where G represents a glycine residue, P represents a proline residue, and X represents an amino acid residue other than glycine), at least an amino acid sequence corresponding to substitution of one glycine residue in one or a plurality of the motif sequences with another amino acid residue, as compared to naturally-occurring spider fibroins.

The second modified fibroin may be a modified fibroin in which a proportion of motif sequences in which the above-mentioned glycine residue is substituted with another amino acid residue is 10% or more with respect to all motif sequences.

The second modified fibroin may be a modified fibroin which has a domain sequence represented by Formula 1: [(A)ₙ motif-REP]ₘ, and has an amino acid sequence in which z/w is 30% or more, 40% or more, 50% or more, or 50.9% or more in a case of defining, as z, a total number of amino acid residues in amino acid sequences consisting of XGX (where X represents an amino acid residue other than glycine) contained in all REP's in a sequence obtained by excluding, from the domain sequence, a sequence from the (A)ₙ motif located furthest to the C-terminal side to the C-terminus of the domain sequence, and defining, as w, a total number of amino acid residues in a sequence obtained by excluding, from the domain sequence, a sequence from the (A)ₙ motif located furthest to the C-terminal side to the C-terminus of the domain sequence. It is sufficient for the number of alanine residues be 83% or more with respect to a total number of amino acid residues in the (A)ₙ motif, but it is preferably 86% or more, more preferably 90% or more, still more preferably 95% or more, and still more preferably 100% (which means that the (A)ₙ motif consists of only alanine residues).

The second modified fibroin is preferably a modified fibroin in which a content ratio of the amino acid sequences consisting of XGX is increased by substituting one glycine residue of a GGX motif with another amino acid residue. In the second modified fibroin, a content ratio of amino acid sequences consisting of GGX in the domain sequence is preferably 30% or less, more preferably 20% or less, still more preferably 10% or less, even still more preferably 6% or less, still further preferably 4% or less, and particularly preferably 2% or less. The content ratio of the amino acid sequences consisting of GGX in the domain sequence can be calculated by the same method as a method (described below) of calculating a content ratio (z/w) of the amino acid sequences consisting of XGX.

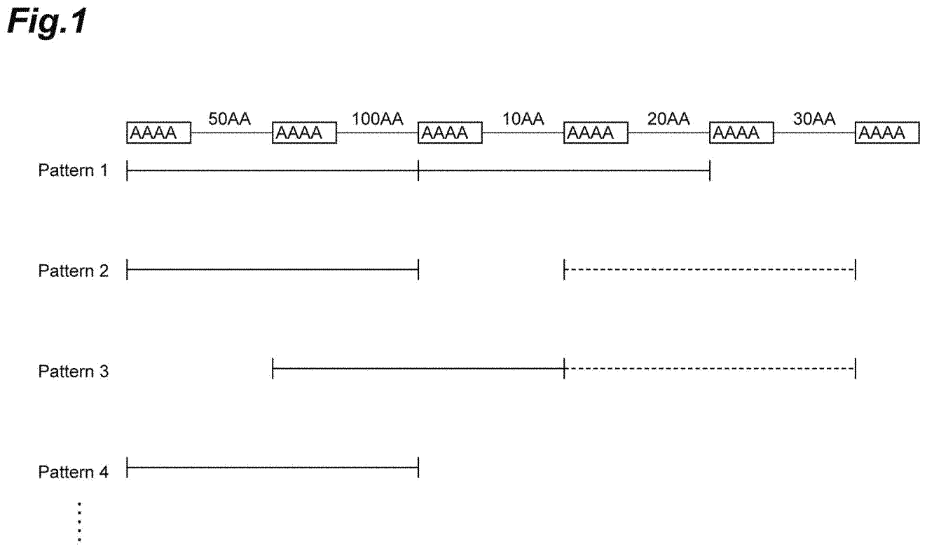

The method of calculating z/w will be described in more detail. First, in a spider fibroin (a modified fibroin or naturally-occurring spider fibroin) having the domain sequence represented by Formula 1: [(A)ₙ motif-REP]ₘ, amino acid sequences consisting of XGX are extracted from all REP's included in a sequence from which a sequence from the (A)ₙ motif located furthest to the C-terminal side from the domain sequence to the C-terminus of the domain sequence. A total number of amino acid residues constituting XGX is z. For example, in a case where 50 amino acid sequences consisting of XGX are extracted (with no overlap), z is 50×3=150. In addition, for example, in a case where the number of X's (central X) contained in XGX is two as in a case of amino acid sequences consisting of XGXGX, a total number of amino acid residues is calculated by subtracting the overlapping portion (where there are 5 amino acid residues in the case of XGXGX). w is a total number of amino acid residues contained in the sequence obtained by excluding, from the domain sequence, the sequence from the (A)ₙ motif located furthest to the C-terminal side to the C-terminus of the domain sequence. For example, in a case of a domain sequence shown in FIG. 1, w is 4+50+4+100+4+10+4+20+4+30=230 (excluding the (A)ₙ motif located furthest to the C-terminal side). Next, z/w (%) can be calculated by dividing z by w.

In the second modified fibroin, z/w is preferably 50.9% or more, more preferably 56.1% or more, still more preferably 58.7% or more, even still more preferably 70% or more, and still further preferably 80% or more. The upper limit of z/w is not particularly limited, but it may be 95% or less, for example.

The second modified fibroin can be obtained, for example, by modification in which at least a part of a base sequence encoding a glycine residue is substituted so as to encode another amino acid residue from a genetic sequence of a cloned naturally-occurring spider fibroin. In this case, one glycine residue in a GGX motif and a GPGXX motif may be selected as a glycine residue to be modified, or substitution may be carried out such that z/w is 50.9% or more. Alternatively, the second modified fibroin can also be obtained, for example, by designing amino acid sequences satisfying the above aspect from amino acid sequences of naturally-occurring spider fibroins and chemically synthesizing nucleic acids encoding the designed amino acid sequence.

In any case, in addition to the modification corresponding to substitution of a glycine residue in REP with another amino acid residue from amino acid sequences of naturally-occurring spider fibroins, modification on an amino acid sequence corresponding to an amino acid sequence in which substitution, deletion, insertion, and/or addition of one or a plurality of amino acid residues has been carried out may be performed.

The above-mentioned other amino acid residue is not particularly limited as long as it is an amino acid residue other than a glycine residue, but it is preferably a hydrophobic amino acid residue such as a valine (V) residue, a leucine (L) residue, an isoleucine (I) residue, a methionine (M) residue, a proline (P) residue, a phenylalanine (F) residue, or a tryptophan (W) residue, or a hydrophilic amino acid residue such as a glutamine (Q) residue, an asparagine (N) residue, a serine (S) residue, a lysine (K) residue, or a glutamic acid (E) residue, among which more preferred is a valine (V) residue, a leucine (L) residue, an isoleucine (I) residue, or a glutamine (Q) residue, and still more preferred is a glutamine (Q) residue.

More specific examples of the second modified fibroin include a modified fibroin (2-i) including an amino acid sequence set forth in SEQ ID NO: 6, SEQ ID NO: 7, SEQ ID NO: 8, or SEQ ID NO: 9; or a modified fibroin (2-ii) including an amino acid sequence having 90% or more sequence identity with the amino acid sequence set forth in SEQ ID NO: 6, SEQ ID NO: 7, SEQ ID NO: 8, or SEQ ID NO: 9.

The modified fibroin (2-i) will be described. The amino acid sequence set forth in SEQ ID NO: 6 is obtained by substituting, with GQX, all GGX's in REP of the amino acid sequence set forth in SEQ ID NO: 10 corresponding to an amino acid sequence of a naturally-occurring spider fibroin. The amino acid sequence set forth in SEQ ID NO: 7 is obtained by deleting the (A)ₙ motif at every other two locations from the N-terminal side to the C-terminal side in the amino acid sequence set forth in SEQ ID NO: 6, and further inserting one [(A)ₙ motif-REP] before the C-terminal sequence. The amino acid sequence set forth in SEQ ID NO: 8 is obtained by inserting two alanine residues at the C-terminal side of each (A)ₙ motif of the amino acid sequence set forth in SEQ ID NO: 7, further substituting some of glutamine (Q) residues with a serine (S) residue, and deleting some of amino acids on the N-terminal side so that a molecular weight is almost the same as that in SEQ ID NO: 7. The amino acid sequence set forth in SEQ ID NO: 9 is an amino acid sequence in which a His tag has been added to the C-terminus of a sequence having four repetitions of a region of 20 domain sequences present in the amino acid sequence set forth in SEQ ID NO: 11 (however, several amino acid residues at the C-terminal side of the region have been substituted).

A value of z/w in the amino acid sequence set forth in SEQ ID NO: 10 (corresponding to that of a naturally-occurring spider fibroin) is 46.8%. Values of z/w in the amino acid sequence set forth in SEQ ID NO: 6, the amino acid sequence set forth in SEQ ID NO: 7, the amino acid sequence set forth in SEQ ID NO: 8, and the amino acid sequence set forth in SEQ ID NO: 9 are respectively 58.7%, 70.1%, 66.1%, and 70.0%. In addition, values of x/y at the Giza ratio (to be described later) of 1:1.8 to 1:11.3 of the amino acid sequences set forth in SEQ ID NO: 10, SEQ ID NO: 6, SEQ ID NO: 7, SEQ ID NO: 8, and SEQ ID NO: 9 are respectively 15.0%, 15.0%, 93.4%, 92.7%, and 89.3%.

The modified fibroin (2-i) may consist of the amino acid sequence set forth in SEQ ID NO: 6, SEQ ID NO: 7, SEQ ID NO: 8, or SEQ ID NO: 9.

The modified fibroin (2-ii) includes an amino acid sequence having 90% or more sequence identity with the amino acid sequence set forth in SEQ ID NO: 6, SEQ ID NO: 7, SEQ ID NO: 8, or SEQ ID NO: 9. The modified fibroin (2-ii) is also a protein including the domain sequence represented by Formula 1: [(A)ₙ motif-REP]ₘ. The sequence identity is preferably 95% or more.

It is preferable that the modified fibroin (2-ii) have 90% or more sequence identity with the amino acid sequence set forth in SEQ ID NO: 6, SEQ ID NO: 7, SEQ ID NO: 8, or SEQ ID NO: 9, and have z/w of 50.9% or more in the case of defining, as z, a total number of amino acid residues in amino acid sequences consisting of XGX contained in REP (where X represents an amino acid residue other than glycine), and defining, as w, a total number of amino acid residues of REP in the domain sequence.

The second modified fibroin may include a tag sequence at either or both of the N-terminus and C-terminus. Thereby, it is possible to isolate, immobilize, detect, and visualize the modified fibroin.

Examples of tag sequences include an affinity tag utilizing specific affinity (bonding properties, affinity) for other molecules. Specific examples of affinity tags include a histidine tag (His tag). The His tag is a short peptide in which about 4 to 10 histidine residues are arranged. Because it has a property of specifically binding to metal ions such as nickel, it can be used for isolation of modified fibroins by chelating metal chromatography. Specific examples of tag sequences include an amino acid sequence set forth in SEQ ID NO: 12 (amino acid sequence including a His tag sequence and a hinge sequence).

In addition, it is also possible to use a tag sequence such as glutathione-S-transferase (GST) that specifically binds to glutathione or a maltose binding protein (MBP) that specifically binds to maltose.

Furthermore, an “epitope tag” utilizing an antigen-antibody reaction can also be used. An antibody against an epitope can be bonded by adding a peptide (epitope) showing antigenicity as a tag sequence. Examples of epitope tags include a HA (peptide sequence of hemagglutinin of influenza virus) tag, a myc tag, a FLAG tag, and the like. The modified fibroin can be easily purified with high specificity by utilizing the epitope tag.

It is also possible to use a modified fibroin in which a tag sequence is cleaved with a specific protease. It is also possible to recover the modified fibroin in which the tag sequence is cleaved by treating a protein adsorbed via the tag sequence with protease.

More specific examples of the second modified fibroin including a tag sequence include a modified fibroin (2-iii) including an amino acid sequence set forth in SEQ ID NO: 13, SEQ ID NO: 11, SEQ ID NO: 14, or SEQ ID NO: 15; or a modified fibroin (2-iv) including an amino acid sequence having 90% or more sequence identity with the amino acid sequence set forth in SEQ ID NO: 13, SEQ ID NO: 11, SEQ ID NO: 14, or SEQ ID NO: 15.

Amino acid sequences set forth in SEQ ID NO: 16, SEQ ID NO: 17, SEQ ID NO: 13, SEQ ID NO: 11, SEQ ID NO: 14, and SEQ ID NO: 15 are amino acid sequences in which the amino acid sequence set forth in SEQ ID NO: 12 (including a His tag sequence and a hinge sequence) is added at the N-terminus of the amino acid sequences set forth in SEQ ID NO: 10, SEQ ID NO: 18, SEQ ID NO: 6, SEQ ID NO: 7, SEQ ID NO: 8, and SEQ ID NO: 9, respectively.

The modified fibroin (2-iii) may consist of the amino acid sequence set forth in SEQ ID NO: 13, SEQ ID NO: 11, SEQ ID NO: 14, or SEQ ID NO: 15.

The modified fibroin (2-iv) includes an amino acid sequence having 90% or more sequence identity with the amino acid sequence set forth in SEQ ID NO: 13, SEQ ID NO: 11, SEQ ID NO: 14, or SEQ ID NO: 15. The modified fibroin (2-iv) is also a protein including the domain sequence represented by Formula 1: [(A)ₙ motif-REP]ₘ. The sequence identity is preferably 95% or more.

It is preferable that the modified fibroin (2-iv) have 90% or more sequence identity with the amino acid sequence set forth in SEQ ID NO: 13, SEQ ID NO: 11, SEQ ID NO: 14, or SEQ ID NO: 15, and have z/w of 50.9% or more in the case of defining, as z, a total number of amino acid residues in amino acid sequences consisting of XGX contained in REP (where X represents an amino acid residue other than glycine), and defining, as w, a total number of amino acid residues of REP in the domain sequence.

The second modified fibroin may include a secretory signal for releasing protein produced in a recombinant protein production system to the outside of a host. A sequence of the secretory signal can be appropriately set depending on the types of host.

In the modified fibroin in which a content of (A)ₙ motifs has been reduced (third modified fibroin), a domain sequence thereof has an amino acid sequence in which a content of (A)ₙ motifs has been reduced, as compared to naturally-occurring spider fibroins. It can be said that the domain sequence of the third modified fibroin has an amino acid sequence corresponding to an amino acid sequence in which at least one or a plurality of (A)ₙ motifs have been deleted, as compared to naturally-occurring spider fibroins.

The third modified fibroin may have an amino acid sequence corresponding to an amino acid sequence in which 10% to 40% of (A)ₙ motifs have been deleted from an amino acid sequence of a naturally-occurring spider fibroin.

The third modified fibroin may be a modified fibroin in which a domain sequence thereof has an amino acid sequence corresponding to an amino acid sequence in which at least one (A)ₙ motif per one to three (A)ₙ motifs has been deleted from the N-terminal side to the C-terminal side, as compared to naturally-occurring spider fibroins.

The third modified fibroin may be a modified fibroin in which a domain sequence thereof has an amino acid sequence corresponding to an amino acid sequence in which at least deletion of two consecutive (A)ₙ motifs and deletion of one (A)ₙ motif have been carried out repeatedly in this order from the N-terminal side to the C-terminal side, as compared to naturally-occurring spider fibroins.

The third modified fibroin may be a modified fibroin in which a domain sequence thereof has an amino acid sequence corresponding to an amino acid sequence in which at least (A)ₙ motifs at every other two positions have been deleted from the N-terminal side to the C-terminal side.

The third modified fibroin may be a modified fibroin which has a domain sequence represented by Formula 1: [(A)ₙ motif-REP]ₘ, and has an amino acid sequence in which x/y is 20% or more, 30% or more, 40% or more, or 50% or more, in a case of defining, as 1, the number of amino acid residues in REP having a smaller number of amino acid residues, which is the number obtained by sequentially comparing the number of amino acid residues in REP's of two adjacent [(A)ₙ motif-REP] units from the N-terminal side to the C-terminal side, defining, as x, a maximum value of a total value obtained by adding all the number of amino acid residues in the two adjacent [(A)ₙ motif-REP] units at which a ratio of the number of amino acid residues in the other REP is 1.8 to 11.3, and defining, as y, a total number of amino acid residues of the domain sequence. It is sufficient for the number of alanine residues be 83% or more with respect to a total number of amino acid residues in the (A)ₙ motif, but it is preferably 86% or more, more preferably 90% or more, still more preferably 95% or more, and still more preferably 100% (which means that the (A)ₙ motif consists of only alanine residues).

A method of calculating x/y will be described in more detail with reference to FIG. 1. FIG. 1 shows a domain sequence excluding an N-terminal sequence and a C-terminal sequence from a spider fibroin. This domain sequence has a sequence of (A)ₙ motif-first REP (50 amino acid residues)-(A)ₙ motif-second REP (100 amino acid residues)-(A)ₙ motif-third REP (10 amino acid residues)-(A)ₙ motif-fourth REP (20 amino acid residues)-(A)ₙ motif-fifth REP (30 amino acid residues)-(A)ₙ motif from the N-terminal side (left side).

The two adjacent [(A)ₙ motif-REP] units are sequentially selected from the N-terminal side to the C-terminal side so as not to overlap. In this case, an unselected [(A)ₙ motif-REP] unit may exist. FIG. 1 shows a pattern 1 (a comparison between the first REP and the second REP and a comparison between the third REP and the fourth REP), a pattern 2 (a comparison between the first REP and the second REP and a comparison between the fourth REP and the fifth REP), a pattern 3 (a comparison between the second REP and the third REP and a comparison between the fourth REP and the fifth REP), and a pattern 4 (a comparison between the first REP and the second REP). There are other selection methods other than the above-mentioned selection.

Next, for each pattern, the number of amino acid residues of each of the REP's in the selected two adjacent [(A)ₙ motif-REP] units is compared. The comparison is carried out by obtaining a ratio of the number of amino acid residues of the other REP in a case where the number of amino acid residues in REP having a smaller number of amino acid residues is defined as 1. For example, in a case of comparing the first REP (50 amino acid residues) and the second REP (100 amino acid residues), a ratio of the number of amino acid residues of the second REP is 100/50=2 in a case where the number of amino acid residues of the first REP which has a smaller number of amino acid residues is defined as 1. Similarly, in a case of comparing the fourth REP (20 amino acid residues) and the fifth REP (30 amino acid residues), a ratio of the number of amino acid residues of the fifth REP is 30/20=1.5 in a case where the number of amino acid residues of the fourth REP which has a smaller number of amino acid residues is defined as 1.

In FIG. 1, a solid line indicates a combination of [(A)ₙ motif-REP] units in which a ratio of the number of amino acid residues of the other REP is 1.8 to 11.3 in a case where the number of amino acid residues of REP which has a smaller number of amino acid residues is defined as 1. Hereinafter, such a ratio is referred to as a Giza ratio. A broken line indicates a combination of [(A)ₙ motif-REP] units in which a ratio of the number of amino acid residues of the other REP is less than 1.8 or more than 11.3 in a case where the number of amino acid residues REP which has a smaller number of amino acid residues is defined as 1.

In each pattern, the number of all amino acid residues of two adjacent [(A)ₙ motif-REP] units indicated by solid lines (including not only the number of amino acid residues of REP's but also the number of amino acid residues of (A)ₙ motifs) is combined. Then, total values thus combined are compared, and a total value of the pattern at which the total value is a maximum (a maximum value of the total value) is defined as x. In the example shown in FIG. 1, a total value of the pattern 1 is a maximum.

Next, x/y (%) can be calculated by dividing x by the total number y of amino acid residues of the domain sequence.

In the third modified fibroin, x/y is preferably 50% or more, more preferably 60% or more, still more preferably 65% or more, even still more preferably 70% or more, still further preferably 75% or more, and particularly preferably 80% or more. The upper limit of x/y is not particularly limited, and it may be 100% or less, for example. In a case where a Giza ratio is 1:1.9 to 1:11.3, x/y is preferably 89.6% or more; in a case where a Giza ratio is 1:1.8 to 1:3.4, x/y is preferably 77.1% or more; in a case where a Giza ratio is 1:1.9 to 1:8.4, x/y is preferably 75.9% or more; and in a case where a Giza ratio is 1:1.9 to 1:4.1, x/y is preferably 64.2% or more.

In a case where the third modified fibroin is a modified fibroin in which at least seven (A)ₙ motifs which are present in plural in the domain sequence are composed of only alanine residues, x/y is preferably 46.4% or more, is more preferably 50% or more, is even more preferably 55% or more, is still even more preferably 60% or more, is still even more preferably 70% or more, and is particularly preferably 80% or more. The upper limit of x/y is not particularly limited, and may be 100% or less.

The third modified fibroin can be obtained by, for example, deleting one or a plurality of sequences encoding the (A)ₙ motif from a genetic sequence of a cloned naturally-occurring spider fibroin such that x/y is 64.2% or more. Alternatively, the third modified fibroin can also be obtained, for example, by designing amino acid sequences corresponding to an amino acid sequence in which one or a plurality of (A)ₙ motifs have been deleted such that x/y is 64.2% or more from amino acid sequences of naturally-occurring spider fibroins and chemically synthesizing nucleic acids encoding the designed amino acid sequence. In any case, in addition to the modification corresponding to deletion of (A)ₙ motifs from amino acid sequences of naturally-occurring spider fibroins, modification on an amino acid sequence corresponding to an amino acid sequence in which substitution, deletion, insertion, and/or addition of one or a plurality of amino acid residues has been carried out may be performed.

More specific examples of the third modified fibroin include a modified fibroin (3-i) including an amino acid sequence set forth in SEQ ID NO: 18, SEQ ID NO: 7, SEQ ID NO: 8, or SEQ ID NO: 9; or a modified fibroin (3-ii) including an amino acid sequence having 90% or more sequence identity with the amino acid sequence set forth in SEQ ID NO: 18, SEQ ID NO: 7, SEQ ID NO: 8, or SEQ ID NO: 9.

The modified fibroin (3-i) will be described. The amino acid sequence set forth in SEQ ID NO: 18 is obtained by deleting the (A)ₙ motif at every other two locations from the N-terminal side to the C-terminal side in the amino acid sequence set forth in SEQ ID NO: 10 corresponding to an amino acid sequence of a naturally-occurring spider fibroin, and further inserting one [(A)ₙ motif-REP] before the C-terminal sequence. The amino acid sequence set forth in SEQ ID NO: 7 is obtained by substituting, with GQX, all GGX's in REP of the amino acid sequence set forth in SEQ ID NO: 18. The amino acid sequence set forth in SEQ ID NO: 8 is obtained by inserting two alanine residues at the C-terminal side of each (A)ₙ motif of the amino acid sequence set forth in SEQ ID NO: 7, further substituting some of glutamine (Q) residues with a serine (S) residue, and deleting some of amino acids on the N-terminal side so that a molecular weight is almost the same as that in SEQ ID NO: 7. The amino acid sequence set forth in SEQ ID NO: 9 is an amino acid sequence in which a His tag has been added to the C-terminus of a sequence having four repetitions of a region of 20 domain sequences present in the amino acid sequence set forth in SEQ ID NO: 11 (however, several amino acid residues at the C-terminal side of the region have been substituted).

A value of x/y in a Giza ratio of 1:1.8 to 1:11.3 of the amino acid sequence set forth in SEQ ID NO: 10 (corresponding to an amino acid sequence of a naturally-occurring spider fibroin) is 15.0%. Values of x/y in the amino acid sequence set forth in SEQ ID NO: 18 and the amino acid sequence set forth in SEQ ID NO: 7 are both 93.4%. A value of x/y in the amino acid sequence set forth in SEQ ID NO: 8 is 92.7%. A value of x/y in the amino acid sequence set forth in SEQ ID NO: 9 is 89.3%. Values of z/w in the amino acid sequences set forth in SEQ ID NO: 10, SEQ ID NO: 18, SEQ ID NO: 7, SEQ ID NO: 8, and SEQ ID NO: 9 are respectively 46.8%, 56.2%, 70.1%, 66.1%, and 70.0%.

The modified fibroin (3-i) may consist of the amino acid sequence set forth in SEQ ID NO: 18, SEQ ID NO: 7, SEQ ID NO: 8, or SEQ ID NO: 9.

The modified fibroin (3-ii) includes an amino acid sequence having 90% or more sequence identity with the amino acid sequence set forth in SEQ ID NO: 18, SEQ ID NO: 7, SEQ ID NO: 8, or SEQ ID NO: 9. The modified fibroin (3-ii) is also a protein including the domain sequence represented by Formula 1: [(A)ₙ motif-REP]ₘ. The sequence identity is preferably 95% or more.

The modified fibroin (3-ii) preferably has 90% or more sequence identity with the amino acid sequence set forth in SEQ ID NO: 18, SEQ ID NO: 7, SEQ ID NO: 8, or SEQ ID NO: 9, and has an amino acid sequence in which x/y is 64.2% or more, in a case of defining, as 1, the number of amino acid residues in REP having a smaller number of amino acid residues, which is the number obtained by sequentially comparing the number of amino acid residues in REP's of two adjacent [(A)ₙ motif-REP] units from the N-terminal side to the C-terminal side, defining, as x, a maximum value of a total value obtained by adding all the number of amino acid residues in the two adjacent [(A)ₙ motif-REP] units at which a ratio of the number of amino acid residues in the other REP is 1.8 to 11.3 (a Giza ratio of 1:1.8 to 1:11.3), and defining, as y, a total number of amino acid residues of the domain sequence.

The third modified fibroin may include the above-mentioned tag sequence at either or both of the N-terminus and the C-terminus.

More specific examples of the third modified fibroin including a tag sequence may be a modified fibroin (3-iii) including an amino acid sequence set forth in SEQ ID NO: 17, SEQ ID NO: 11, SEQ ID NO: 14, or SEQ ID NO: 15; or a modified fibroin (3-iv) including an amino acid sequence having 90% or more sequence identity with the amino acid sequence set forth in SEQ ID NO: 17, SEQ ID NO: 11, SEQ ID NO: 14, or SEQ ID NO: 15.

Amino acid sequences set forth in SEQ ID NO: 16, SEQ ID NO: 17, SEQ ID NO: 13, SEQ ID NO: 11, SEQ ID NO: 14, and SEQ ID NO: 15 are amino acid sequences in which the amino acid sequence set forth in SEQ ID NO: 12 (including a His tag sequence and a hinge sequence) is added at the N-terminus of the amino acid sequences set forth in SEQ ID NO: 10, SEQ ID NO: 18, SEQ ID NO: 6, SEQ ID NO: 7, SEQ ID NO: 8, and SEQ ID NO: 9, respectively.

The modified fibroin (3-iii) may consist of the amino acid sequence set forth in SEQ ID NO: 17, SEQ ID NO: 11, SEQ ID NO: 14, or SEQ ID NO: 15.

The modified fibroin (3-iv) includes an amino acid sequence having 90% or more sequence identity with the amino acid sequence set forth in SEQ ID NO: 17, SEQ ID NO: 11, SEQ ID NO: 14, or SEQ ID NO: 15. The modified fibroin (3-iv) is also a protein including the domain sequence represented by Formula 1: [(A)ₙ motif-REP]ₘ. The sequence identity is preferably 95% or more.

The modified fibroin (3-iv) preferably has 90% or more sequence identity with the amino acid sequence set forth in SEQ ID NO: 17, SEQ ID NO: 11, SEQ ID NO: 14, or SEQ ID NO: 15, and has an amino acid sequence in which x/y is 64.2% or more, in a case of defining, as 1, the number of amino acid residues in REP having a smaller number of amino acid residues, which is the number obtained by sequentially comparing the number of amino acid residues in REP's of two adjacent [(A)ₙ motif-REP] units from the N-terminal side to the C-terminal side, defining, as x, a maximum value of a total value obtained by adding all the number of amino acid residues in the two adjacent [(A)ₙ motif-REP] units at which a ratio of the number of amino acid residues in the other REP is 1.8 to 11.3, and defining, as y, a total number of amino acid residues of the domain sequence.

The third modified fibroin may include a secretory signal for releasing protein produced in a recombinant protein production system to the outside of a host. A sequence of the secretory signal can be appropriately set depending on the types of host.

The modified fibroin in which a content of glycine residues and a content of (A)ₙ motifs have been reduced (fourth modified fibroin) is a modified fibroin in which a domain sequence thereof has an amino acid sequence in which a content of (A)ₙ motifs have been reduced and a content of glycine residues has further been reduced, as compared to naturally-occurring spider fibroins. It can be said that the domain sequence of the fourth modified fibroin has an amino acid sequence corresponding to an amino acid sequence in which at least one or a plurality of (A)ₙ motifs have been deleted and at least one or a plurality of glycine residues in REP have further been substituted with another amino acid residue, as compared to naturally-occurring spider fibroins. In other words, the fourth modified fibroin is a modified fibroin having characteristics of both the modified fibroin in which a content of glycine residues has been reduced (the second modified fibroin), and the modified fibroin in which a content of (A)ₙ motifs has been reduced (the third modified fibroin). Specific aspects and the like thereof are as described for the second modified fibroin and the third modified fibroin.

More specific examples of the fourth modified fibroin include a modified fibroin (4-i) including an amino acid sequence set forth in SEQ ID NO: 7, SEQ ID NO: 8, or SEQ ID NO: 9, or a modified fibroin (4-ii) including an amino acid sequence having 90% or more sequence identity with the amino acid sequence set forth in SEQ ID NO: 7, SEQ ID NO: 8, or SEQ ID NO: 9. Specific aspects of the modified fibroin including the amino acid sequence set forth in SEQ ID NO: 7, SEQ ID NO: 8, or SEQ ID NO: 9 are as described above.

A modified fibroin including a domain sequence having a region locally having a large hydrophobicity index (fifth modified fibroin) may be a modified fibroin in which the domain sequence thereof includes an amino acid sequence corresponding to an amino acid sequence which has a region locally having a large hydrophobicity index and in which one or a plurality of amino acid residues in REP is substituted with an amino acid residue having a large hydrophobicity index and/or one or a plurality of amino acid residues having a large hydrophobicity index is inserted into the REP, as compared to naturally-occurring spider fibroins.

The region locally having a large hydrophobicity index is preferably composed of 2 to 4 consecutive amino acid residues.

The above-mentioned amino acid residue having a large hydrophobicity index is more preferably an amino acid residue selected from isoleucine (I), valine (V), leucine (L), phenylalanine (F), cysteine (C), methionine (M), and alanine (A).

The fifth modified fibroin may have, in addition to the modification corresponding to substitution of one or a plurality of amino acid residues in REP with an amino acid residue having a large hydrophobicity index, and/or insertion of one or a plurality of amino acid residues having a large hydrophobicity index into the REP, as compared to naturally-occurring spider fibroins, a modification on an amino acid sequence corresponding to an amino acid sequence in which substitution, deletion, insertion, and/or addition of one or a plurality of amino acid residues has been carried out, as compared to naturally-occurring spider fibroins.

The fifth modified fibroin can be obtained by substituting one or a plurality of hydrophilic amino acid residues in REP (for example, amino acid residues having a negative hydrophobicity index) with hydrophobic amino acid residues (for example, amino acid residues having a positive hydrophobicity index) from a genetic sequence of a cloned naturally-occurring spider fibroin, and/or by inserting one or a plurality of hydrophobic amino acid residues into REP. In addition, the fifth modified fibroin can be obtained by, for example, designing an amino acid sequence corresponding to an amino acid sequence in which substitution of one or a plurality of hydrophilic amino acid residues in REP with hydrophobic amino acid residues is carried out from an amino acid sequence of a naturally-occurring spider fibroin, and/or insertion of one or a plurality of hydrophobic amino acid residues into REP is carried out; and chemically synthesizing nucleic acids encoding the designed amino acid sequence. In any case, in addition to the modification corresponding to substitution of one or a plurality of hydrophilic amino acid residues in REP with hydrophobic amino acid residues from an amino acid sequence of a naturally-occurring spider fibroin, and/or insertion of one or a plurality of hydrophobic amino acid residues into REP, further modification on the amino acid sequence corresponding to substitution, deletion, insertion and/or addition of one or a plurality of amino acid residues may be carried out.

The fifth modified fibroin includes the domain sequence represented by Formula 1: [(A)ₙ motif-REP]ₘ, and may include an amino acid sequence having p/q of 6.2% or more in a case of defining, as p, a total number of amino acid residues contained in a region in which an average value of hydrophobicity indices of four consecutive amino acid residues is 2.6 or more, in all REP's included in a sequence obtained by excluding, from the domain sequence, a sequence from the (A)ₙ motif located furthest to the C-terminal side to the C-terminus of the domain sequence, and defining, as q, a total number of amino acid residues contained in the sequence obtained by excluding, from the domain sequence, the sequence from the (A)ₙ motif located furthest to the C-terminal side to the C-terminus of the domain sequence.

Regarding the hydrophobicity index of amino acid residues, known indices (Hydropathy index: Kyte J, & Doolittle R (1982) “A simple method for displaying the hydropathic character of a protein,” J. Mol. Biol., 157, pp. 105-132) are used. Specifically, the hydrophobicity index (hydropathy index, hereinafter will be referred to as “HI”) of each amino acid is as shown in Table 1.

TABLE 1

| Amino acid | HI |
|---|---|
| Isoleucine (Ile) | 4.5 |
| Valine (Val) | 4.2 |
| Leucine (Leu) | 3.8 |
| Phenylalanine (Phe) | 2.8 |
| Cysteine (Cys) | 2.5 |
| Methionine (Met) | 1.9 |
| Alanine (Ala) | 1.8 |
| Glycine (Gly) | −0.4 |
| Threonine (Thr) | −0.7 |
| Serine (Ser) | −0.8 |
| Tryptophan (Trp) | −0.9 |
| Tyrosine (Tyr) | −1.3 |
| Proline (Pro) | −1.6 |
| Histidine (His) | −3.2 |
| Asparagine (Asn) | −3.5 |
| Asparaginic acid (Asp) | −3.5 |
| Glutamine (Gln) | −3.5 |
| Glutamic acid (Glu) | −3.5 |
| Lysine (Lys) | −3.9 |
| Arginine (Arg) | −4.5 |

A method of calculating p/q will be described in more detail. In calculation, the sequence obtained by excluding, from the domain sequence represented by Formula 1: [(A)ₙ motif-REP]ₘ, the sequence from the (A)ₙ motif located furthest to the C-terminal side to the C-terminus of the domain sequence is used (hereinafter referred to as a “sequence A”). First, an average value of hydrophobicity indices of four consecutive amino acid residues in all REP's included in the sequence A is calculated. The average value of the hydrophobicity indices is obtained by dividing a sum of HI's of each amino acid residue contained in the four consecutive amino acid residues by 4 (the number of amino acid residues). The average value of the hydrophobicity indices is obtained for all four consecutive amino acid residues (where each amino acid residue is used to calculate an average 1 to 4 times). Next, a region in which the average value of the hydrophobicity indices of the four consecutive amino acid residues is 2.6 or more is identified. Even in a case where a certain amino acid residue corresponds to a plurality of “four consecutive amino acid residues having an average value of the hydrophobicity indices of 2.6 or more,” this amino acid residue is contained in the region as one amino acid residue. In addition, a total number of amino acid residues contained in the region is p. Furthermore, a total number of amino acid residues contained in the sequence A is q.

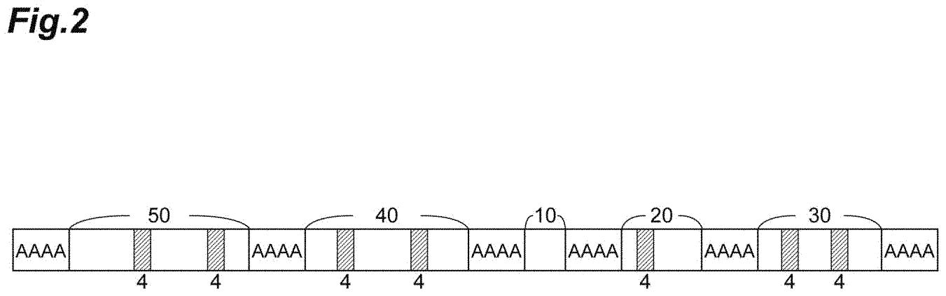

For example, in a case where “four consecutive amino acid residues having an average value of the hydrophobicity indices of 2.6 or more” are extracted at 20 locations (with no overlap), 20 times four consecutive amino acid residues (with no overlap) are contained in the region in which the average value of the hydrophobicity indices of the four consecutive amino acid residues is 2.6 or more, and therefore p is 20×4=80. In addition, for example, in a case where only one amino acid residue overlaps in two “four consecutive amino acid residues having an average value of the hydrophobicity indices of 2.6 or more,” seven amino acid residues are contained in the region in which the average value of the hydrophobicity indices of the four consecutive amino acid residues is 2.6 or more (p=2×4−1=7, where “−1” is a subtraction of the overlapping portion). For example, in a case of a domain sequence shown in FIG. 2, seven “four consecutive amino acid residues having an average value of the hydrophobicity indices of 2.6 or more” are present without overlapping, and therefore p is 7×4=28. In addition, for example, in the case of the domain sequence shown in FIG. 2, q is 4+50+4+40+4+10+4+20+4+30=170 (not including (A)ₙ motif present at the end of the C-terminal side). Next, p/q (%) can be calculated by dividing p by q. In the case of FIG. 2, 28/170=16.47%.

In the fifth modified fibroin, p/q is preferably 6.2% or more, more preferably 7% or more, still more preferably 10% or more, even still more preferably 20% or more, and still further preferably 30% or more. The upper limit of p/q is not particularly limited, but it may be 45% or less, for example.

The fifth modified fibroin can be obtained by, for example, modifying an amino acid sequence of a cloned naturally-occurring spider fibroin to an amino acid sequence including a region locally having a large hydrophobicity index, such that the above conditions for p/q are satisfied by substituting one or a plurality of hydrophilic amino acid residues in REP (for example, amino acid residues having a negative hydrophobicity index) with hydrophobic amino acid residues (for example, amino acid residues having a positive hydrophobicity index), and/or by inserting one or a plurality of hydrophobic amino acid residues into REP. Alternatively, the fifth modified fibroin can also be obtained, for example, by designing an amino acid sequence satisfying the conditions for p/q from an amino acid sequence of a naturally-occurring spider fibroin, and chemically synthesizing nucleic acids encoding the designed amino acid sequence. In any case, in addition to the modification corresponding to substitution of one or a plurality of amino acid residues in REP with amino acid residues having a large hydrophobicity index, and/or insertion of one or a plurality of amino acid residues having a large hydrophobicity index into REP as compared to naturally-occurring spider fibroins, further modification corresponding to substitution, deletion, insertion, and/or addition of one or a plurality of amino acid residues may be carried out.

The above-mentioned amino acid residue having a large hydrophobicity index is not particularly limited, but it is preferably isoleucine (I), valine (V), leucine (L), phenylalanine (F), cysteine (C), methionine (M), and alanine (A), and is more preferably valine (V), leucine (L), and isoleucine (I).

Specific examples of the fifth modified fibroin include a modified fibroin (5-i) including the amino acid sequence set forth in SEQ ID NO: 19, SEQ ID NO: 20, or SEQ ID NO: 21, or a modified fibroin (5-ii) including an amino acid sequence having 90% or more sequence identity with the amino acid sequence set forth in SEQ ID NO: 19, SEQ ID NO: 20, or SEQ ID NO: 21.

The modified fibroin (5-i) will be described. The amino acid sequence set forth in SEQ ID NO: 22 is obtained by carrying out deletion in an amino acid sequence in which consecutive alanine residues are present in (A)ₙ motif of a naturally-occurring spider fibroin such that the number of consecutive alanine residues becomes 5. The amino acid sequence set forth in SEQ ID NO: 19 is obtained by inserting an amino acid sequence (VLI) consisting of three amino acid residues at two locations of every other REP in the amino acid sequence set forth in SEQ ID NO: 22, and deleting some of amino acids at the C-terminal side so that a molecular weight becomes almost the same as that of the amino acid sequence set forth in SEQ ID NO: 22. The amino acid sequence set forth in SEQ ID NO: 23 is obtained by inserting two alanine residues at the C-terminal side of each (A)ₙ motif in the amino acid sequence set forth in SEQ ID NO: 22, further substituting some of glutamine (Q) residues with a serine (S) residue, and deleting some of amino acids on the C-terminal side so that a molecular weight becomes almost the same as that of the amino acid sequence set forth in SEQ ID NO: 22. The amino acid sequence set forth in SEQ ID NO: 20 is obtained by inserting an amino acid sequence (VLI) consisting of three amino acid residues at one location of every other REP in the amino acid sequence set forth in SEQ ID NO: 23. The amino acid sequence set forth in SEQ ID NO: 21 is obtained by inserting an amino acid sequence (VLI) consisting of three amino acid residues at two locations of every other REP in the amino acid sequence set forth in SEQ ID NO: 23.

The modified fibroin (5-i) may consist of the amino acid sequence set forth in SEQ ID NO: 19, SEQ ID NO: 20, or SEQ ID NO: 21.

The modified fibroin (5-ii) includes an amino acid sequence having 90% or more sequence identity with the amino acid sequence set forth in SEQ ID NO: 19, SEQ ID NO: 20, or SEQ ID NO: 21. The modified fibroin (5-ii) is also a protein including the domain sequence represented by Formula 1: [(A)ₙ motif-REP]ₘ. The sequence identity is preferably 95% or more.

The modified fibroin (5-ii) has 90% or more sequence identity with the amino acid sequence set forth in SEQ ID NO: 19, SEQ ID NO: 20, or SEQ ID NO: 21, and preferably has p/q of 6.2% or more in a case of defining, as p, a total number of amino acid residues contained in a region in which an average value of hydrophobicity indices of four consecutive amino acid residues is 2.6 or more in all REP's included in a sequence obtained by excluding, from the domain sequence, a sequence from the (A)ₙ motif located furthest to the C-terminal side to the C-terminus of the domain sequence, and defining, as q, a total number of amino acid residues contained in the sequence obtained by excluding, from the domain sequence, the sequence from the (A)ₙ motif located furthest to the C-terminal side to the C-terminus of the domain sequence.

The fifth modified fibroin may include a tag sequence at either or both of the N-terminus and C-terminus.

More specific examples of the fifth modified fibroin including a tag sequence include a modified fibroin (5-iii) including an amino acid sequence set forth in SEQ ID NO: 24, SEQ ID NO: 25, or SEQ ID NO: 26, or a modified fibroin (5-iv) including an amino acid sequence having 90% or more sequence identity with the amino acid sequence set forth in SEQ ID NO: 24, SEQ ID NO: 25, or SEQ ID NO: 26.

The amino acid sequences set forth in SEQ ID NO: 24, SEQ ID NO: 25, and SEQ ID NO: 26 are amino acid sequences in which the amino acid sequence set forth in SEQ ID NO: 12 (including a His tag sequence and a hinge sequence) is added at each of the N-terminus of the amino acid sequences set forth in SEQ ID NO: 19, SEQ ID NO: 20, and SEQ ID NO: 21.

The modified fibroin (5-iii) may consist of the amino acid sequence set forth in SEQ ID NO: 24, SEQ ID NO: 25, or SEQ ID NO: 26.

The modified fibroin (5-iv) includes an amino acid sequence having 90% or more sequence identity with the amino acid sequence set forth in SEQ ID NO: 24, SEQ ID NO: 25, or SEQ ID NO: 26. The modified fibroin (5-iv) is also a protein including the domain sequence represented by Formula 1: [(A)ₙ motif-REP]ₘ. The sequence identity is preferably 95% or more.

The modified fibroin (5-iv) has 90% or more sequence identity with the amino acid sequence set forth in SEQ ID NO: 24, SEQ ID NO: 25, or SEQ ID NO: 26, and preferably has p/q of 6.2% or more in a case of defining, as p, a total number of amino acid residues contained in a region in which an average value of hydrophobicity indices of four consecutive amino acid residues is 2.6 or more in all REP's included in a sequence obtained by excluding, from the domain sequence, a sequence from the (A)ₙ motif located furthest to the C-terminal side to the C-terminus of the domain sequence, and defining, as q, a total number of amino acid residues contained in the sequence obtained by excluding, from the domain sequence, the sequence from the (A)ₙ motif located furthest to the C-terminal side to the C-terminus of the domain sequence.

The fifth modified fibroin may include a secretory signal for releasing protein produced in a recombinant protein production system to the outside of a host. A sequence of the secretory signal can be appropriately set depending on the types of host.

A modified fibroin having a domain sequence in which a content of glutamine residues has been reduced (sixth modified fibroin) has an amino acid sequence in which a content of glutamine residues has been reduced, as compared to naturally-occurring spider fibroins.

The sixth modified fibroin preferably contains at least one motif selected from a GGX motif and a GPGXX motif in an amino acid sequence of REP.

In a case where the sixth modified fibroin contains a GPGXX motif in REP, a content rate of the GPGXX motif is generally 1% or more, it may be 5% or more, and it is preferably 10% or more. The upper limit of the content rate of the GPGXX motif is not particularly limited, and it may be 50% or less or 30% or less.

In the present specification, a “content rate of the GPGXX motif” is a value calculated by the following method.

In a spider fibroin having the domain sequence represented by Formula 1: [(A)ₙ motif-REP]ₘ or Formula 2: [(A)ₙ motif-REP]ₘ-(A)ₙ motif, a content rate of the GPGXX motif is calculated as s/t in a case of defining, as s, in all REP's included in a sequence obtained by excluding, from the domain sequence, a sequence from the (A)ₙ motif located furthest to the C-terminal side to the C-terminus of the domain sequence, a number obtained by multiplying a total number of GPGXX motifs contained in the region by three (that is, corresponding to a total number of G's and P's in the GPGXX motif), and defining, as t, a total number of amino acid residues in all REP's included in a sequence obtained by excluding, from the domain sequence, a sequence from the (A)ₙ motif located furthest to the C-terminal side to the C-terminus of the domain sequence and further a sequence of the (A)ₙ motif located furthest to the C-terminal side.

In calculation of a content rate of the GPGXX motif, “the sequence from the (A)ₙ motif located furthest to the C-terminal side to the C-terminus of the domain sequence” (a sequence corresponding to REP) sometimes includes a sequence having a low correlation with a sequence characteristic of spider fibroins, and the reason for targeting “the sequence obtained by excluding, from the domain sequence, the sequence from the (A)ₙ motif located furthest to the C-terminal side to the C-terminus of the domain sequence” is to eliminate an influence of a small m (that is, a short domain sequence) on calculation results for a content rate of the GPGXX motif. In a case where the “GPGXX motif” is located at the C-terminus of REP, and even when “XX” is “AA,” it is regarded as a “GPGXX motif”

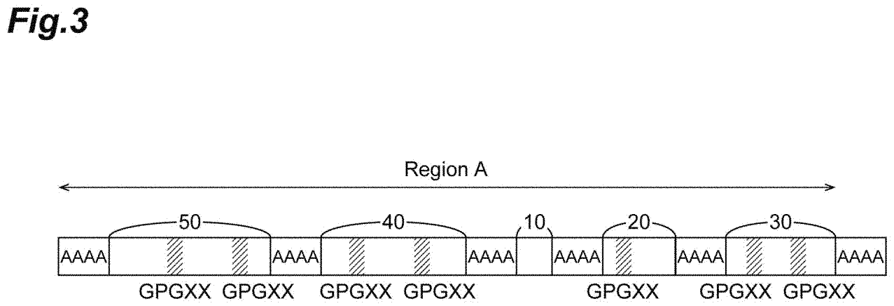

FIG. 3 is a schematic diagram showing a domain sequence of a spider fibroin. A method of calculating a content rate of the GPGXX motif will be specifically described with reference to FIG. 3. First, in the domain sequence (that is the “[(A)ₙ motif-REP]ₘ-(A)ₙ motif” type) of the spider fibroin shown in FIG. 3, since all REP's are included in a “sequence obtained by excluding, from the domain sequence, a sequence from the (A)ₙ motif located furthest to the C-terminal side to the C-terminus of the domain sequence” (a sequence shown as a “region A” in FIG. 3), the number of GPGXX motifs for calculating s is 7, and therefore s is 7×3=21. Similarly, since all REP's are included in the “sequence obtained by excluding, from the domain sequence, the sequence from the (A)ₙ motif located furthest to the C-terminal side to the C-terminus of the domain sequence” (the sequence shown as a “region A” in FIG. 3), a total number t of amino acid residues in the all REP's from which (A)ₙ motifs are further excluded from the sequence is 50+40+10+20+30=150. Then, s/t (%) can be calculated by dividing s by t, and it is 21/150=14.0% in the case of the fibroin of FIG. 3.

A content rate of glutamine residues in the sixth modified fibroin is preferably 9% or less, more preferably 7% or less, even more preferably 4% or less, and particularly preferably 0%.

In the present specification, a “content rate of glutamine residues” is a value calculated by the following method.

In a spider fibroin having the domain sequence represented by Formula 1: [(A)ₙ motif-REP]ₘ or Formula 2: [(A)ₙ motif-REP]ₘ-(A)ₙ motif, a content rate of glutamine residues is calculated as u/t in a case of defining, as u, in all REP's included in the sequence (the sequence corresponding to the “region A” in FIG. 3) obtained by excluding, from the domain sequence, a sequence from the (A)ₙ motif located furthest to the C-terminal side to the C-terminus of the domain sequence, a total number of glutamine residues contained in the region, and defining, as t, a total number of amino acid residues in all REP's included in a sequence obtained by excluding, from the domain sequence, a sequence from the (A)ₙ motif located furthest to the C-terminal side to the C-terminus of the domain sequence and further a sequence of the (A)ₙ motif located furthest to the C-terminal side. In the calculation of a content rate of glutamine residues, the reason for targeting the “sequence obtained by excluding, from the domain sequence, the sequence from the (A)ₙ motif located furthest to the C-terminal side to the C-terminus of the domain sequence” is the same as the above-described reason.

In the sixth modified fibroin, a domain sequence thereof may have an amino acid sequence corresponding to an amino acid sequence in which one or a plurality of glutamine residues in REP have been deleted or substituted with another amino acid residue, as compared with naturally-occurring spider fibroins.

The “other amino acid residue” may be any amino acid residue other than a glutamine residue, but it is preferably an amino acid residue having a larger hydrophobicity index than that of a glutamine residue. Hydrophobicity indices of amino acid residues are shown in Table 1.

As shown in Table 1, examples of amino acid residues having a larger hydrophobicity index than that of glutamine residues include amino acid residues selected from isoleucine (I), valine (V), leucine (L), phenylalanine (F), cysteine (C), methionine (M), alanine (A), glycine (G), threonine (T), serine (S), tryptophan (W), tyrosine (Y), proline (P), and histidine (H). Among them, an amino acid residue selected from isoleucine (I), valine (V), leucine (L), phenylalanine (F), cysteine (C), methionine (M), and alanine (A) is more preferable, and an amino acid residue selected from isoleucine (I), valine (V), leucine (L), and phenylalanine (F) is even more preferable.

In the sixth modified fibroin, a hydrophobicity of REP is preferably −0.8% or more, more preferably −0.7% or more, still more preferably 0% or more, even still more preferably 0.3% or more, and still further preferably 0.4% or more. The upper limit of the hydrophobicity of REP is not particularly limited, and it may be 1.0 or less, or 0.7 or less.

In the present specification, a “hydrophobicity of REP” is a value calculated by the following method.

In a spider fibroin having the domain sequence represented by Formula 1: [(A)ₙ motif-REP]ₘ or Formula 2: [(A)ₙ motif-REP]ₘ-(A)ₙ motif, a hydrophobicity of REP is calculated as v/t in a case of defining, as v, in all REP's included in the sequence (the sequence corresponding to the “region A” in FIG. 3) obtained by excluding, from the domain sequence, a sequence from the (A)ₙ motif located furthest to the C-terminal side to the C-terminus of the domain sequence, a sum of hydrophobicity indices of each of amino acid residues in the region and defining, as t, a total number of amino acid residues in all REP's included in a sequence obtained by excluding, from the domain sequence, a sequence from the (A)ₙ motif located furthest to the C-terminal side to the C-terminus of the domain sequence and further a sequence of the (A)ₙ motif located furthest to the C-terminal side. In the calculation of a hydrophobicity of REP, the reason for targeting the “sequence obtained by excluding, from the domain sequence, the sequence from the (A)ₙ motif located furthest to the C-terminal side to the C-terminus of the domain sequence” is the same as the above-described reason.

The sixth modified fibroin includes a domain sequence having a modification corresponding to deletion of one or a plurality of glutamine residues in REP, and/or substitution of one or a plurality of glutamine residues in REP with another amino acid residue, and the domain sequence may further have a modification corresponding to an amino acid sequence in which substitution, deletion, insertion, and/or addition of one or a plurality of amino acid residues has been carried out, as compared to naturally-occurring spider fibroins.

The sixth modified fibroin can be obtained by, for example, deleting one or a plurality of glutamine residues in REP from a cloned genetic sequence of a naturally-occurring spider fibroin, and/or substituting one or a plurality of glutamine residues in REP with another amino acid residue. In addition, the sixth modified fibroin can be obtained by, for example, designing an amino acid sequence corresponding to an amino acid sequence in which one or a plurality of glutamine residues in REP have been deleted from an amino acid sequence of a naturally-occurring spider fibroin, and/or one or a plurality of glutamine residues in REP have been substituted with another amino acid residue; and chemically synthesizing nucleic acids encoding the designed amino acid sequence.

More specific examples of the sixth modified fibroin include a modified fibroin (6-i) including an amino acid sequence set forth in SEQ ID NO: 27, SEQ ID NO: 28, SEQ ID NO: 29, SEQ ID NO: 30, SEQ ID NO: 31, SEQ ID NO: 32, SEQ ID NO: 33, or SEQ ID NO: 43, or a modified fibroin (6-ii) including an amino acid sequence having 90% or more sequence identity with the amino acid sequence set forth in SEQ ID NO: 27, SEQ ID NO: 28, SEQ ID NO: 29, SEQ ID NO: 30, SEQ ID NO: 31, SEQ ID NO: 32, SEQ ID NO: 33, or SEQ ID NO: 43.

The modified fibroin (6-i) will be described.

The amino acid sequence set forth in SEQ ID NO: 7 (Met-PRT410) is an amino acid sequence in which amino acids have been modified for improvement in productivity, such as setting the number of consecutive alanine residues to 5 in an amino acid sequence in which alanine residues in (A)ₙ motif are consecutive, based on a base sequence and an amino acid sequence of a naturally-occurring fibroin, Nephila clavipes (GenBank Accession No.: P46804.1, GI: 1174415). Meanwhile, because a glutamine residue (Q) is not modified in Met-PRT410, a content rate of glutamine residues is about the same as a content rate of glutamine residues in a naturally-occurring fibroin.

The amino acid sequence set forth in SEQ ID NO: 27 (M_PRT888) is obtained by substituting all QQ's in Met-PRT410 (SEQ ID NO: 7) with VL.

The amino acid sequence set forth in SEQ ID NO: 28 (M_PRT965) is obtained by substituting all QQ's in Met-PRT410 (SEQ ID NO: 7) with TS and substituting the remaining Q with A.

The amino acid sequence set forth in SEQ ID NO: 29 (M_PRT889) is obtained by substituting all QQ's in Met-PRT410 (SEQ ID NO: 7) with VL and substituting the remaining Q with I.

The amino acid sequence set forth in SEQ ID NO: 30 (M_PRT916) is obtained by substituting all QQ's in Met-PRT410 (SEQ ID NO: 7) with VI and substituting the remaining Q with L.

The amino acid sequence set forth in SEQ ID NO: 31 (M_PRT918) is obtained by substituting all QQ's in Met-PRT410 (SEQ ID NO: 7) with VF and substituting the remaining Q with I.

The amino acid sequence set forth in SEQ ID NO: 34 (M_PRT525) is an amino acid sequence in which two alanine residues has been inserted into a region (A5) in which alanine residues are consecutive, two domain sequences at the C-terminal side have been deleted such that a molecular weight becomes almost the same as that of Met-PRT410, and 13 locations of glutamine residues (Q) have been substituted with serine residues (S) or proline residues (P) in Met-PRT410 (SEQ ID NO: 7).

The amino acid sequence set forth in SEQ ID NO: 32 (M_PRT699) is obtained by substituting all QQ's in M_PRT525 (SEQ ID NO: 34) with VL.

The amino acid sequence set forth in SEQ ID NO: 33 (M_PRT698) is obtained by substituting all QQ's in M_PRT525 (SEQ ID NO: 34) with VL and substituting the remaining Q with I.

The amino acid sequence set forth in SEQ ID NO: 43 (Met-PRT966) is an amino acid sequence in which all QQ's in the amino acid sequence set forth in SEQ ID NO: 9 (the amino acid sequence before the amino acid sequence set forth in SEQ ID NO: 42 is added to the C-terminus) have been substituted with VF, and the remaining Q has been substituted by I.

In all the amino acid sequences set forth in SEQ ID NO: 27, SEQ ID NO: 28, SEQ ID NO: 29, SEQ ID NO: 30, SEQ ID NO: 31, SEQ ID NO: 32, SEQ ID NO: 33, and SEQ ID NO: 43, a content rate of glutamine residues is 9% or less (Table 2).

TABLE 2

| | Content rate of | Content rate of | Hydrophobicity |
|---|---|---|---|
| Modified fibroin | glutamine residues | GPGXX motifs | index of REP |
| Met-PRT410 | 17.7% | 27.9% | −1.52 |
| (SEQ ID NO: 7) | | | |
| M_PRT888 | 6.3% | 27.9% | −0.07 |
| (SEQ ID NO: 27) | | | |
| M_PRT965 | 0.0% | 27.9% | −0.65 |
| (SEQ ID NO: 28) | | | |
| M_PRT889 | 0.0% | 27.9% | 0.35 |
| (SEQ ID NO: 29) | | | |
| M_PRT916 | 0.0% | 27.9% | 0.47 |
| (SEQ ID NO: 30) | | | |
| M_PRT918 | 0.0% | 27.9% | 0.45 |
| (SEQ ID NO: 31) | | | |
| M_PRT525 | 13.7% | 26.4% | −1.24 |
| (SEQ ID NO: 34) | | | |
| M_PRT699 | 3.6% | 26.4% | −0.78 |
| (SEQ ID NO: 32) | | | |
| M_PRT698 | 0.0% | 26.4% | −0.03 |
| (SEQ ID NO: 33) | | | |
| Met-PRT966 | 0.0% | 28.0% | 0.35 |
| (SEQ ID NO: 43) | | | |

The modified fibroin (6-i) may consist of the amino acid sequence set forth in SEQ ID NO: 27, SEQ ID NO: 28, SEQ ID NO: 29, SEQ ID NO: 30, SEQ ID NO: 31, SEQ ID NO: 32, SEQ ID NO: 33, or SEQ ID NO: 43.

The modified fibroin (6-ii) includes an amino acid sequence having 90% or more sequence identity with the amino acid sequence set forth in SEQ ID NO: 27, SEQ ID NO: 28, SEQ ID NO: 29, SEQ ID NO: 30, SEQ ID NO: 31, SEQ ID NO: 32, SEQ ID NO: 33, or SEQ ID NO: 43. The modified fibroin (6-ii) is also a protein having the domain sequence represented by Formula 1: [(A)ₙ motif-REP]ₘ or Formula 2: [(A)ₙ motif-REP]ₘ-(A)ₙ motif. The sequence identity is preferably 95% or more.

In the modified fibroin (6-ii), a content rate of glutamine residues is preferably 9% or less. In addition, in the modified fibroin (6-ii), a content rate of GPGXX motifs is preferably 10% or more.

The sixth modified fibroin may include a tag sequence at either or both of the N-terminus and C-terminus. Thereby, it is possible to isolate, immobilize, detect, and visualize the modified fibroin.

More specific examples of the sixth modified fibroin having a tag sequence include a modified fibroin (6-iii) including an amino acid sequence set forth in SEQ ID NO: 35, SEQ ID NO: 36, SEQ ID NO: 37, SEQ ID NO: 38, SEQ ID NO: 39, SEQ ID NO: 40, SEQ ID NO: 41, or SEQ ID NO: 44, or a modified fibroin (6-iv) including an amino acid sequence having 90% or more sequence identity with the amino acid sequence set forth in SEQ ID NO: 35, SEQ ID NO: 36, SEQ ID NO: 37, SEQ ID NO: 38, SEQ ID NO: 39, SEQ ID NO: 40, SEQ ID NO: 41, or SEQ ID NO: 44.

Amino acid sequences set forth in SEQ ID NO: 35, SEQ ID NO: 36, SEQ ID NO: 37, SEQ ID NO: 38, SEQ ID NO: 39, SEQ ID NO: 40, SEQ ID NO: 41, and SEQ ID NO: 44 are amino acid sequences in which the amino acid sequence set forth in SEQ ID NO: 12 (including a His tag sequence and a hinge sequence) is added at the N-terminus of the amino acid sequences set forth in SEQ ID NO: 27, SEQ ID NO: 28, SEQ ID NO: 29, SEQ ID NO: 30, SEQ ID NO: 31, SEQ ID NO: 32, SEQ ID NO: 33, and SEQ ID NO: 43, respectively. Because addition of the tag sequence to the N-terminus has been merely carried out, a content rate of glutamine residues does not change, and therefore a content rate of glutamine residues is 9% or less in all the amino acid sequences set forth in SEQ ID NO: 35, SEQ ID NO: 36, SEQ ID NO: 37, SEQ ID NO: 38, SEQ ID NO: 39, SEQ ID NO: 40, SEQ ID NO: 41, and SEQ ID NO: 44 (Table 3).

TABLE 3

| | Content rate of | Content rate of | Hydrophobicity |
|---|---|---|---|
| Modified fibroin | glutamine residues | GPGXX motifs | index of REP |
| PRT888 | 6.3% | 27.9% | −0.07 |
| (SEQ ID NO: 35) | | | |
| PRT965 | 0.0% | 27.9% | −0.65 |
| (SEQ ID NO: 36) | | | |
| PRT889 | 0.0% | 27.9% | 0.35 |
| (SEQ ID NO: 37) | | | |
| PRT916 | 0.0% | 27.9% | 0.47 |
| (SEQ ID NO: 38) | | | |
| PRT918 | 0.0% | 27.9% | 0.45 |
| (SEQ ID NO: 39) | | | |
| PRT699 | 3.6% | 26.4% | −0.78 |
| (SEQ ID NO: 40) | | | |
| PRT698 | 0.0% | 26.4% | −0.03 |
| (SEQ ID NO: 41) | | | |
| PRT966 | 0.0% | 28.0% | 0.35 |
| (SEQ ID NO: 44) | | | |

The modified fibroin (6-iii) may consist of the amino acid sequence set forth in SEQ ID NO: 35, SEQ ID NO: 36, SEQ ID NO: 37, SEQ ID NO: 38, SEQ ID NO: 39, SEQ ID NO: 40, SEQ ID NO: 41, or SEQ ID NO: 44.

The modified fibroin (6-iv) includes an amino acid sequence having 90% or more sequence identity with the amino acid sequence set forth in SEQ ID NO: 35, SEQ ID NO: 36, SEQ ID NO: 37, SEQ ID NO: 38, SEQ ID NO: 39, SEQ ID NO: 40, SEQ ID NO: 41, or SEQ ID NO: 44. The modified fibroin (6-iv) is also a protein having the domain sequence represented by Formula 1: [(A)ₙ motif-REP]ₘ or Formula 2: [(A)ₙ motif-REP]ₘ-(A)ₙ motif. The sequence identity is preferably 95% or more.

In the modified fibroin (6-iv), a content rate of glutamine residues is preferably 9% or less. In addition, in the modified fibroin (6-iv), a content rate of GPGXX motifs is preferably 10% or more.

The sixth modified fibroin may include a secretory signal for releasing protein produced in a recombinant protein production system to the outside of a host. A sequence of the secretory signal can be appropriately set depending on the types of host.

The modified fibroin may be a modified fibroin having at least two or more characteristics in combination among the characteristics of the first modified fibroin, the second modified fibroin, the third modified fibroin, the fourth modified fibroin, the fifth modified fibroin, and the sixth modified fibroin.

The spider silk protein may be a hydrophilic spider silk protein or a hydrophobic spider silk protein. The hydrophobic spider silk protein is a spider silk protein of a case in which a sum of hydrophobicity indices (HI) of all amino acid residues constituting the spider silk protein is obtained, and then a value (average HI) obtained by dividing the sum by a total number of amino acid residues is 0 or more. The hydrophobicity index is as shown in Table 1. In addition, the hydrophilic spider silk protein is a spider silk protein having an average HI of less than 0.

Examples of hydrophobic spider silk proteins include the sixth modified fibroin. More specific examples of hydrophobic spider silk proteins include a spider silk protein having the amino acid sequence set forth in SEQ ID NO: 27, SEQ ID NO: 28, SEQ ID NO: 29, SEQ ID NO: 30, SEQ ID NO: 31, SEQ ID NO: 32, SEQ ID NO: 33, or SEQ ID NO: 43, and a spider silk protein having the amino acid sequence set forth in SEQ ID NO: 35, SEQ ID NO: 37, SEQ ID NO: 38, SEQ ID NO: 39, SEQ ID NO: 40, SEQ ID NO: 41, or SEQ ID NO: 44.

Examples of hydrophilic spider silk proteins include the first modified fibroin, the second modified fibroin, the third modified fibroin, the fourth modified fibroin, and the fifth modified fibroin. More specific examples of hydrophilic spider silk proteins include a spider silk protein having the amino acid sequence set forth in SEQ ID NO: 4; a spider silk protein having the amino acid sequence set forth in SEQ ID NO: 6, SEQ ID NO: 7, SEQ ID NO: 8, or SEQ ID NO: 9; a spider silk protein having the amino acid sequence set forth in SEQ ID NO: 13, SEQ ID NO: 11, SEQ ID NO: 14, or SEQ ID NO: 15; a spider silk protein having the amino acid sequence set forth in SEQ ID NO: 18, SEQ ID NO: 7, SEQ ID NO: 8, or SEQ ID NO: 9; a spider silk protein having the amino acid sequence set forth in SEQ ID NO: 17, SEQ ID NO: 11, SEQ ID NO: 14, or SEQ ID NO: 15; and a spider silk protein having the amino acid sequence set forth in SEQ ID NO: 19, SEQ ID NO: 20, or SEQ ID NO: 21.

The spider silk proteins described above may be used alone or in combination of two or more kinds thereof.

The spider silk protein can be produced by, for example, expressing a nucleic acid in a host transformed with an expression vector having a nucleic acid sequence encoding the spider silk protein and one or a plurality of regulatory sequences operably linked to the nucleic acid sequence.

A method for producing a nucleic acid encoding a spider silk protein is not particularly limited. A nucleic acid can be produced by, for example, a method in which a gene encoding natural spider silk proteins is amplified and cloned by polymerase chain reaction (PCR) or the like; or a method of chemically synthesizing nucleic acids. A method for chemically synthesizing nucleic acids is not particularly limited, and, for example, genes can be chemically synthesized by a method of linking, by PCR or the like, oligonucleotides that are automatically synthesized by AKTA oligopilot plus 10/100 (GE Healthcare Japan Ltd.) or the like, based on amino acid sequence information of spider silk proteins obtained from the NCBI web database and the like. In this case, in order to facilitate purification and/or confirmation of spider silk proteins, a nucleic acid encoding a spider silk protein consisting of an amino acid sequence obtained by adding an amino acid sequence consisting of a start codon and a His10 tag to the N-terminus may be synthesized.

The regulatory sequence is a sequence (for example, a promoter, an enhancer, a ribosome binding sequence, or a transcription termination sequence) that controls the expression of a recombinant protein in a host, and can be appropriately selected depending on the types of hosts. As a promoter, an inducible promoter which functions in host cells and is capable of inducible expression of a target spider silk protein may be used. The inducible promoter is a promoter that can control transcription due to the presence of an inducer (expression inducer), the absence of a repressor molecule, or physical factors such as an increase or decrease in temperature, osmotic pressure, or pH value.

The types of expression vectors such as a plasmid vector, a viral vector, a cosmid vector, a fosmid vector, and an artificial chromosome vector can be appropriately selected depending on the types of hosts. As the expression vector, an expression vector which can autonomously replicate in a host cell or can be incorporated into a chromosome of a host and which contains a promoter at a position capable of transcribing the nucleic acid encoding a spider silk protein is suitably used.

Both prokaryotes and eukaryotes such as yeast, filamentous fungi, insect cells, animal cells, and plant cells can be suitably used as hosts.

In the case where a prokaryote such as a bacterium is used as a host, the expression vector is preferably a vector which is capable of autonomous replication in the prokaryote and, at the same time, includes a promoter, a ribosome binding sequence, a nucleic acid encoding a spider silk protein, and a transcription termination sequence. A gene that controls a promoter may be included.

Examples of prokaryotes include microorganisms belonging to the genus Escherichia, the genus Brevibacillus, the genus Serratia, the genus Bacillus, the genus Microbacterium, the genus Brevibacterium, the genus Corynebacterium, and the genus Pseudomonas. Examples of microorganisms belonging to the genus Escherichia include Escherichia coli and the like. Examples of microorganisms belonging to the genus Brevibacillus include Brevibacillus agri and the like. Examples of microorganisms belonging to the genus Serratia include Serratia liquefaciens and the like. Examples of microorganisms belonging to the genus Bacillus include Bacillus subtilis and the like. Examples of microorganisms belonging to the genus Microbacterium include Microbacterium ammoniaphilum. Examples of microorganisms belonging to the genus Brevibacterium include Brevibacterium divaricatum and the like. Examples of microorganisms belonging to the genus Corynebacterium include Corynebacterium ammoniagenes and the like. Examples of microorganisms belonging to the genus Pseudomonas include Pseudomonas putida and the like.

In a case where a prokaryote is used as a host, examples of vectors into which a nucleic acid encoding a spider silk protein is introduced include pBTrp2 (manufactured by Boehringer Mannheim), pGEX (manufactured by Pharmacia), pUC18, pBluescriptII, pSupex, pET22b, pCold, pUB110, and pNCO2 (Japanese Unexamined Patent Publication No. 2002-238569), and the like.

Examples of eukaryotic hosts include yeast and filamentous fungi (mold and the like). Examples of yeasts include a yeast which belongs to the genus Saccharomyces, the genus Pichia, the genus Schizosaccharomyces, and the like. Examples of filamentous fungi include filamentous fungi belonging to the genus Aspergillus, the genus Penicillium, the genus Trichoderma, and the like.

In a case where a eukaryote is used as a host, examples of vectors into which a nucleic acid encoding a spider silk protein is introduced include YEp13 (ATCC37115), YEp24 (ATCC37051), and the like. As a method for introducing an expression vector into the foregoing host cell, any method can be used as long as it introduces DNA into the host cell. Examples thereof include a method using calcium ions [Proc. Natl. Acad. Sci. USA, 69, 2110 (1972)], an electroporation method, a spheroplast method, a protoplast method, a lithium acetate method, a competent method, and the like.

In addition to direct expression, as a method for expressing a nucleic acid by a host transformed with an expression vector, secretory production, fusion protein expression, or the like can be carried out according to the method described in Molecular Cloning, 2nd edition, and the like.

A spider silk protein can be produced, for example, by culturing a host transformed in a culture medium, producing and accumulating the spider silk protein in the culture medium, and then collecting the spider silk protein from the culture medium. The method for culturing the transformed host in a culture medium can be carried out according to a method commonly used for culturing a host.

In the case where the host is a prokaryote such as Escherichia coli or a eukaryote such as yeast, any of a natural medium and a synthetic medium may be used as a culture medium as long as it contains a carbon source, a nitrogen source, inorganic salts and the like which can be assimilated by the host and it is capable of efficiently culturing the host.

As the carbon source, any carbon source that can be assimilated by the host may be used. Examples of carbon sources that can be used include carbohydrates such as glucose, fructose, sucrose, and molasses, starch and starch hydrolyzates containing them, organic acids such as acetic acid and propionic acid, and alcohols such as ethanol and propanol.

Examples of nitrogen sources that can be used include ammonium salts of inorganic or organic acids such as ammonia, ammonium chloride, ammonium sulfate, ammonium acetate and ammonium phosphate, other nitrogen-containing compounds, peptone, meat extract, yeast extract, corn steep liquor, casein hydrolyzate, soybean cake and soybean cake hydrolyzate, various fermented microbial cells and digested products thereof.

As inorganic salts, for example, it is possible to use potassium dihydrogen phosphate, dipotassium phosphate, magnesium phosphate, magnesium sulfate, sodium chloride, ferrous sulfate, manganese sulfate, copper sulfate, and calcium carbonate.

Culture of a prokaryote such as Escherichia coli or a eukaryote such as yeast can be carried out under aerobic conditions such as shaking culture or deep aeration stirring culture. A culture temperature is, for example, 15° C. to 40° C. A culture time is usually 16 hours to 7 days. It is preferable to maintain a pH of a culture medium during the culture at 3.0 to 9.0. A pH of the culture medium can be adjusted using an inorganic acid, an organic acid, an alkali solution, urea, calcium carbonate, ammonia, or the like.

In addition, antibiotics such as ampicillin and tetracycline may be added to the culture medium as necessary during the culture. In the case of culturing a microorganism transformed with an expression vector using an inducible promoter as a promoter, an inducer may be added to the medium as necessary. For example, in the case of culturing a microorganism transformed with an expression vector using a lac promoter, isopropyl-β-D-thiogalactopyranoside or the like is used, and in the case of culturing a microorganism transformed with an expression vector using a trp promoter, indole acrylic acid or the like may be added to the medium.

A spider silk protein produced by a transformed host can be isolated and purified by a method commonly used for protein isolation and purification. For example, in the case where the spider silk protein is expressed in a dissolved state in cells, the host cells are recovered by centrifugation after completion of the culture, suspended in an aqueous buffer solution, and then disrupted using an ultrasonicator, a French press, a Manton-Gaulin homogenizer, a Dyno-Mill, or the like to obtain a cell-free extract. From the supernatant obtained by centrifuging the cell-free extract, a purified preparation can be obtained by a method commonly used for protein isolation and purification, that is, a solvent extraction method, a salting-out method using ammonium sulfate or the like, a desalting method, a precipitation method using an organic solvent, an anion exchange chromatography method using a resin such as diethylaminoethyl (DEAE)-Sepharose or DIAION HPA-75 (manufactured by Mitsubishi Kasei Kogyo Kabushiki Kaisha), an cation exchange chromatography method using a resin such as S-Sepharose FF (Pharmacia Corporation), a hydrophobic chromatography method using a resin such as butyl sepharose or phenyl sepharose, a gel filtration method using a molecular sieve, an affinity chromatography method, a chromatofocusing method, an electrophoresis method such as isoelectric focusing or the like, alone or in combination thereof.

As the chromatography, column chromatography using phenyl-TOYOPEARL (available from Tosoh Corporation), DEAE-TOYOPEARL (available from Tosoh Corporation), and Sephadex G-150 (available from Pharmacia Biotech Inc.) is preferably used.

In the case where the spider silk protein is expressed by the formation of an insoluble matter in the cell, similarly, the host cells are recovered, disrupted and centrifuged to recover the insoluble matter of the spider silk protein as a precipitated fraction. The recovered insoluble matter of the spider silk protein can be solubilized with a protein denaturing agent. After this operation, a purified preparation of the spider silk protein can be obtained by the same isolation and purification method as described above.

In the case where the spider silk protein is secreted extracellularly, the spider silk protein can be recovered from the culture supernatant. That is, a culture supernatant is obtained by treating the culture by a technique such as centrifugation, and a purified preparation can be obtained from the culture supernatant by using the same isolation and purification method as described above.

A content of spider silk protein may be 1% by mass or more, 2% by mass or more, 4% by mass or more, 7% by mass or more, 10% by mass or more, or 15% by mass or more, and may be 40% by mass or less, 35% by mass or less, 30% by mass or less, or 25 mass % or less, with respect to a total amount of a spinning raw liquid.

<Solvent>

A solvent used for the spinning raw liquid can be used without particular limitation as long as it can dissolve or disperse spider silk proteins.

Examples of solvents include organic solvents such as dimethyl sulfoxide (DMSO), N,N-dimethylformamide (DMF), N-methylpyrrolidone (NMP), N,N-dimethylacetamide (DMAc), hexafluoro-2-propanol (HFIP), hexafluoroacetone (HFA), and formic acid. In addition, the solvent may be water to which a dissolution promoter to be described later is added. The solvent may be used alone or in combination of two or more kinds thereof.

The solvent is preferably at least one selected from the group consisting of dimethyl sulfoxide, formic acid, and a solvent obtained by adding a dissolution promoter to these solvents.

(Dissolution Promoter)

The spinning raw liquid (dope solution) may further contain a dissolution promoter. The spinning raw liquid can be easily prepared by incorporating the dissolution promoter.

The dissolution promoter may be, for example, an inorganic salt composed of the following Lewis acid and Lewis base. Examples of the Lewis base include an oxo acid ion (nitrate ion, perchlorate ion, or the like), a metal oxo acid ion (permanganate ion or the like), a halide ion, a thiocyanate ion, and a cyanate ion. Examples of the Lewis acid include a metal ion such as an alkali metal ion or an alkaline earth metal ion, a polyatomic ion such as an ammonium ion, and a complex ion. Examples of inorganic salts include lithium salts such as lithium chloride, lithium bromide, lithium iodide, lithium nitrate, lithium perchlorate, and lithium thiocyanate; calcium salts such as calcium chloride, calcium bromide, calcium iodide, calcium nitrate, calcium perchlorate, and calcium thiocyanate; iron salts such as iron chloride, iron bromide, iron iodide, iron nitrate, iron perchlorate, and iron thiocyanate; aluminum salts such as aluminum chloride, aluminum bromide, aluminum iodide, aluminum nitrate, aluminum perchlorate, and aluminum thiocyanate; potassium salts such as potassium chloride, potassium bromide, potassium iodide, potassium nitrate, potassium perchlorate, and potassium thiocyanate; sodium salts such as sodium chloride, sodium bromide, sodium iodide, sodium nitrate, sodium perchlorate, and sodium thiocyanate; zinc salts such as zinc chloride, zinc bromide, zinc iodide, zinc nitrate, zinc perchlorate, and zinc thiocyanate; magnesium salts such as magnesium chloride, magnesium bromide, magnesium iodide, magnesium nitrate, magnesium perchlorate, and magnesium thiocyanate; barium salts such as barium chloride, barium bromide, barium iodide, barium nitrate, barium perchlorate, and barium thiocyanate; and strontium salts such as strontium chloride, strontium bromide, strontium iodide, strontium nitrate, strontium perchlorate, and strontium thiocyanate. These inorganic salts are used as a dissolution promoter for spider silk proteins with respect to formic acid. Since the spinning raw liquid contains the dissolution promoter (the above-mentioned inorganic salt), the spider silk protein can be dissolved in the spinning raw liquid at a high concentration. Accordingly, it is expected that production efficiency of protein fibers will be further improved, that quality of protein fibers and physical properties such as stress will be improved, and the like. The inorganic salt may be at least one selected from the group consisting of lithium chloride and calcium chloride. The dissolution promoter may be used alone or in combination of two or more kinds thereof.

A content of the dissolution promoter may be 0.1% by mass or more, 1% by mass or more, 4% by mass or more, 7% by mass or more, 10% by mass or more, or 15% by mass or more, and may be 20% by mass or less, 16% by mass or less, 12% by mass or less, or 9% by mass or less, with respect to a total amount of the spinning raw liquid.

(Various Additives)

The spinning raw liquid may further contain various additives as necessary. Examples of additives include plasticizers, leveling agents, cross-linking agents, crystal nucleating agents, antioxidants, ultraviolet absorbers, coloring agents, fillers, and synthetic resins. A content of the additive may be 50 parts by mass or less with respect to 100 parts by mass of a total amount of protein in the spinning raw liquid.

The spinning raw liquid is obtained by dissolving spider silk protein in a solvent. The spinning raw liquid may further contain alcohol as long as the effect of the present invention is not impaired.

A viscosity of the spinning raw liquid may be appropriately set according to the spinning method, and it can be set to 100 to 15,000 centipoise (cP) at 35° C., for example. The viscosity of the spinning raw liquid can be measured, for example, by using an “EMS viscometer” (trade name, manufactured by Kyoto Electronics Manufacturing Co., Ltd.).

<Coagulation Liquid>

The coagulation liquid contains a ketone. Thereby, excellent gloss can be realized. In addition, it can be expected that a maximum draw ratio of drawing by a washing bath is improved, yarn running in the washing bath is stable without sagging, and thus yarn passing in an adjacent direction is not interfered with and step passability is improved, and stress is improved. The coagulation liquid may further contain at least one selected from the group consisting of a solvent, a dissolution promoter, and water contained in the spinning raw liquid.

In the present specification, the “ketone” is a compound represented by Formula (1): R₁—C(═O)—R₂. In Formula (1), R₁ and R₂ each independently represent a linear or branched alkyl group having 1 to 6 carbon atoms. R₁ and R₂ may form a ring via a single bond. Specific examples of ketones include acetone, methyl ethyl ketone, methyl isobutyl ketone, diisobutyl ketone, and cyclohexanone. The ketone may be used alone or in combination of two or more kinds thereof.

The ketone contained in the coagulation liquid may be a water-soluble ketone. In the present specification, the “water-soluble ketone” means a ketone that dissolves by 15 g or more (20° C.) in 100 mL of water.

Examples of water-soluble ketones include acetone and methyl ethyl ketone. As the water-soluble ketone, it is preferable to use inexpensive acetone because then production costs can be further reduced. In addition, it is preferable to use acetone having a low boiling point because a coagulation solvent can be easily recovered by distillation.

A content of the ketone is preferably 60% by mass or more, more preferably 70% by mass or more, even more preferably 80% by mass or more, still more preferably 90% by mass or more, and particularly preferably 95% by mass or more with respect to a total amount of the coagulation liquid. In particular, in a case where the spinning raw liquid contains a hydrophilic spider silk protein, a content of ketone is preferably 70% by mass or more, more preferably 80% by mass or more, even more preferably 90% by mass, and particularly preferably 95% by mass or more with respect to the total amount of the coagulation liquid. The coagulation liquid may be composed of only a ketone.

In a case where the coagulation liquid contains water, a content of water is preferably 30% by mass or less with respect to the total amount of the coagulation liquid. A content of water may be 20% by mass or less, or 10% by mass or less, with respect to the total amount of the coagulation liquid.

In a case where the coagulation liquid contains formic acid, a content of formic acid is preferably 40% by mass or less with a total content of ketone and formic acid being 100% by mass. A content of formic acid may be 30% by mass or less, 20% by mass or less, or 10% by mass or less, with the total content of ketone and formic acid being 100% by mass. In a case where the content of formic acid is within the above range, stress of a manufactured protein fiber is further increased. The term stress in the present specification means a value (unit: MPa) obtained by dividing the maximum load (N) until a protein fiber is fractured by an external tensile force in a fiber axis direction by a cross-sectional area of the protein fiber (calculated from a diameter of the protein fiber) (m²).

The coagulation liquid may contain a dissolution promoter. As the dissolution promoter in the coagulation liquid, for example, the above-mentioned inorganic salt may be used. In addition, the coagulation liquid may contain a dissolution promoter by using a solvent containing the dissolution promoter. The coagulation liquid may contain an inorganic salt, and may contain at least one selected from the group consisting of lithium chloride and calcium chloride. The dissolution promoter in the coagulation liquid may be used alone or in combination of two or more kinds thereof

<Spinning Step>

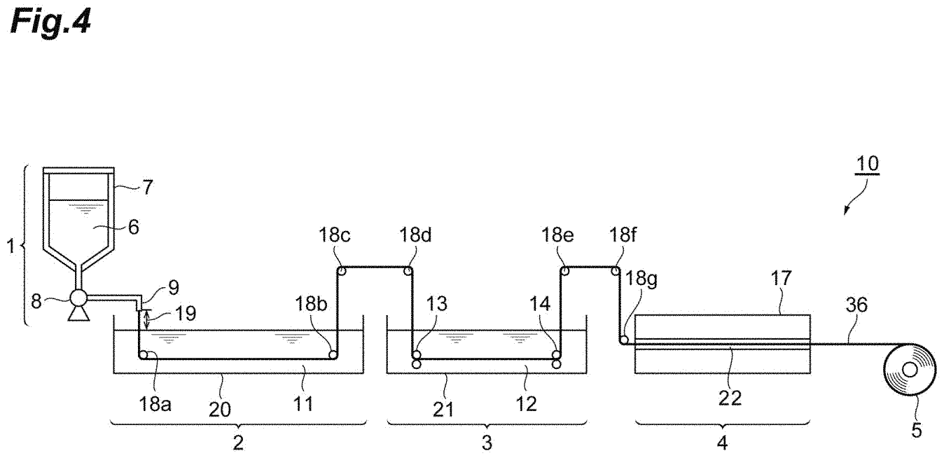

In the spinning step, the above-mentioned spinning raw liquid is brought into contact with the above-mentioned coagulation liquid, and thereby the spider silk protein is coagulated. A method for manufacturing a protein fiber of the present embodiment including the spinning step can be carried out by using, for example, a spinning apparatus shown in FIG. 4.

FIG. 4 is an explanatory view schematically showing an example of the spinning apparatus for manufacturing protein fibers. A spinning apparatus 10 shown in FIG. 4 is an example of a spinning apparatus for dry-wet-type spinning, and has an extrusion apparatus 1, a coagulation bath 20, a washing bath 21 (drawing bath), and a drying apparatus 4 in this order from the upstream side.

The extrusion apparatus 1 has a storage tank 7, in which a spinning raw liquid (dope solution) 6 is stored. A coagulation liquid 11 is stored in the coagulation bath 20. The spinning raw liquid 6 is extruded from a nozzle 9, which is provided to have an air gap 19 between the coagulation liquid 11, by a gear pump 8 attached at a lower end of the storage tank 7. The extruded spinning raw liquid 6 is supplied (introduced) into the coagulation liquid 11 in the coagulation bath 20 through the air gap 19. A solvent is removed from the spinning raw liquid in the coagulation liquid 11 to coagulate spider silk proteins. The coagulated spider silk protein is guided to the washing bath 21 and washed with a washing solution 12 in the washing bath 21, and then sent to the drying apparatus 4 by a first nip roller 13 and a second nip roller 14 installed in the washing bath 21. At this time, for example, when a rotational speed of the second nip roller 14 is set to be faster than a rotational speed of the first nip roller 13, a protein fiber 36 drawn at a magnification corresponding to a rotational speed ratio is obtained. The protein fiber drawn in the washing solution 12 is separated from the inside of the washing bath 21 to be dried while passing through the drying apparatus 4. Thereafter, the fiber is wound up by a winder. Accordingly, the protein fiber is obtained as a wound product 5 which is finally wound around the winder by the spinning apparatus 10. 18a to 18g are yarn guides.

A temperature of the coagulation liquid 11 is not particularly limited, but it may be 40° C. or lower, 30° C. or lower, 25° C. or lower, 20° C. or lower, 10° C. or lower, or 5° C. or lower. It is preferably 0° C. or higher from the viewpoint of workability, cooling cost, and the like. The temperature of the coagulation liquid 11 can be adjusted by using, for example, the spinning apparatus 10 including the coagulation bath 20 having a heat exchanger inside and a cooling circulation device. For example, the temperature within the above range by exchanging heat between the coagulation liquid 11 and the heat exchanger can be adjusted by flowing a medium cooled to a predetermined temperature by the cooling circulation device to the heat exchanger installed in the coagulation bath. In this case, more efficient cooling can be achieved by circulating a solvent used as a medium for the coagulation liquid 11.

A plurality of coagulation baths in which the coagulation liquid is stored may be provided.

The coagulated spider silk protein may be separated from the coagulation bath or the washing bath and then wound up around the winder as it is, or may be passed through the drying apparatus to be dried and then wound around the winder.

A distance that the coagulated spider silk protein passes through the coagulation liquid may be any distance as long as desolvation can be efficiently performed, it may be determined depending on an extrusion speed (discharge speed) or the like of the spinning raw liquid from the nozzle. A retention time of the coagulated spider silk protein (or the spinning raw liquid) in the coagulation liquid may be determined according to the distance that the coagulated spider silk protein passes through the coagulation liquid, the extrusion speed of the spinning raw liquid from the nozzle, and the like.

<Drawing Step>

The method for manufacturing a protein fiber of the present embodiment may further include a step of drawing the coagulated spider silk protein (drawing step). The drawing step may be performed in the coagulation bath 20 or the washing bath 21, for example. The drawing step can also be carried out in air.

The drawing performed in the washing bath 21 may be so-called wet heat drawing performed in warm water, in a solution in which an organic solvent or the like is added to warm water, or the like. A temperature for wet heat drawing is preferably 50° C. to 90° C. When the temperature is 50° C. or higher, a fine pore diameter of the yarn can be stably reduced. When the temperature is 90° C. or lower, the temperature can be easily set, and thereby spinning stability is improved. The temperature is more preferably 75° C. to 85° C.

As a draw ratio of the coagulated spider silk protein in the drawing step becomes higher, yarn running in the bath becomes stable, and thereby step passability is improved without interfering the adjacent running yarn. In the method for manufacturing a protein fiber of the present embodiment, the maximum draw ratio can be improved, and a range for controlling the draw ratio can be widened, and thereby a fiber diameter of the protein fiber can be controlled.

The lower limit of the final draw ratio is preferably any one of more than 1 time, 2 times or more, 3 times or more, 4 times or more, 5 times or more, 6 times or more, 7 times or more, 8 times or more, or 9 times or more a draw ratio of the undrawn yarn (or pre-drawn yarn). The upper limit is preferably 40 times or less, 30 times or less, 20 times or less, 15 times or less, 14 times or less, 13 times or less, 12 times or less, 11 times or less, or 10 times or less.

### EXAMPLES

Hereinafter, the present invention will be described more specifically with reference to examples. However, the present invention is not limited to the following examples.

### (1) Synthesis of Nucleic Acid Encoding Spider Silk Protein and Construction of Expression Vector

Based on a base sequence and an amino acid sequence of a fibroin (GenBank Accession Number: P46804.1, GI: 1174415) derived from Nephila clavipes, spider silk proteins (hereinafter, also referred to as “PRT799” and “PRT966,” respectively) having amino acid sequences set forth in SEQ ID NO: 15 and SEQ ID NO: 44 were designed.

The amino acid sequence set forth in SEQ ID NO: 9 (Met-PRT799) is an amino acid sequence in which amino acid residues have been substituted, inserted, and deleted and an amino acid sequence (hinge sequence and His tag) set forth in SEQ ID NO: 42 has been added to the C-terminus in the amino acid sequence of the fibroin derived from Nephila clavipes for the purpose of improving productivity. The amino acid sequence set forth in SEQ ID NO: 43 (Met-PRT966) is an amino acid sequence in which all QQ's in the amino acid sequence set forth in SEQ ID NO: 9 (the amino acid sequence before the amino acid sequence set forth in SEQ ID NO: 42 is added to the C-terminus) have been substituted by VF, and the remaining Q has been substituted by I for the purpose of improving hydrophobicity. The amino acid sequence set forth in SEQ ID NO: 15 (PRT799) is an amino acid sequence in which the amino acid sequence set forth in SEQ ID NO: 12 (amino acid sequence including a His tag sequence and a hinge sequence) has been added to the N-terminus of the amino acid sequence set forth in SEQ ID NO: 9. The amino acid sequence set forth in SEQ ID NO: 44 (PRT966) is an amino acid sequence in which the amino acid sequence set forth in SEQ ID NO: 12 has been added to the N-terminus of the amino acid sequence set forth in SEQ ID NO: 43.

A nucleic acid encoding a spider silk protein having the designed amino acid sequences set forth in SEQ ID NO: 15 and SEQ ID NO: 44 was synthesized. In the nucleic acid, an NdeI site was added to the 5′ end and an EcoRI site was added downstream of the stop codon. These two kinds of nucleic acids were cloned into a cloning vector (pUC118). Thereafter, the same nucleic acid was cleaved by a restriction enzyme treatment with NdeI and EcoRI, and then recombined into a protein expression vector pET-22b(+) to obtain an expression vector.

### (2) Expression of Spider Silk Protein

Escherichia coli BLR (DE3) was transformed with the expression vector obtained in (1). The transformed Escherichia coli was cultured in 2 mL of an LB medium containing ampicillin for 15 hours. The culture solution was added to 100 mL of a seed culture medium (Table 4) containing ampicillin so that the OD₆₀₀ was 0.005. A temperature of the culture solution was maintained at 30° C., and flask culture was carried out (for about 15 hours) until the OD₆₀₀ reached 5, thereby obtaining a seed culture solution.

TABLE 4
Medium for seed culture (per 1 L when starting culture)

| | |
|---|---|
| Glucose | 5 g |
| KH₂PO₄ | 4 g |
| K₂HPO₄ | 10 g |
| Yeast Extract | 6 g |

A medium for seed culture was prepared by adding ampicillin so that a final concentration became 100 mg/L.

The seed culture solution was added to a jar fermenter, to which 500 mL of a production medium (Table 5) had been added so that the OD₆₀₀ was 0.05, to inoculate the transformed Escherichia coli. The culture was carried out while maintaining a temperature of the culture solution at 37° C. and maintaining a pH thereof constant at 6.9. Furthermore, a concentration of oxygen dissolved in the culture solution was maintained at 20% of a saturated concentration of dissolved oxygen.

TABLE 5
Produced medium (per 1 L when starting culture)

| | | |
|---|---|---|
| Glucose | 12 | g |
| KH₂PO₄ | 9 | g |
| MgSO₄•7H₂O | 2.4 | g |
| Yeast Extract | 15 | g |
| FeSO₄•7H₂O | 40 | mg |
| MnSO₄•5H₂O | 40 | mg |
| CaCl₂•2H₂O | 40 | mg |
| GD-113 (antifoaming agent) | 0.1 | mL |

Immediately after glucose in the production medium was completely consumed, a feed solution (455 g/1 L of glucose and 120 g/1 L of Yeast Extract) was added at a rate of 1 mL/min. The culture was carried out while maintaining a temperature of the culture solution at 37° C. and maintaining a pH thereof constant at 6.9. Furthermore, a concentration of oxygen dissolved in the culture solution was maintained at 20% of a saturated concentration of dissolved oxygen, and the culture was carried out for 20 hours. Thereafter, 1 M isopropyl-β-thiogalactopyranoside (IPTG) was added to the culture solution so that a final concentration became 1 mM to induce the expression of the target spider silk protein. 20 hours after addition of IPTG, the culture solution was centrifuged to recover the bacterial cells. SDS-PAGE was carried out using the bacterial cells prepared from the culture solution before the addition of IPTG and after the addition of IPTG, and the expression of the target spider silk protein was confirmed by a band having a size of the target spider silk protein appeared according to the addition of IPTG

### (3) Purification of Spider Silk Protein

The bacterial cells recovered 2 hours after the addition of IPTG were washed with 20 mM Tris-HCl buffer solution (pH 7.4). The bacterial cells after washing were suspended in 20 mM Tris-HCl buffer solution (pH 7.4) containing about 1 mM PMSF, and the cells were disrupted with a high-pressure homogenizer (available from GEA Niro Soavi SpA). The disrupted cells were centrifuged to obtain a precipitate. The obtained precipitate was washed with 20 mM Tris-HCl buffer solution (pH 7.4) until reaching a high purity. The precipitate after washing was suspended in 8 M guanidine buffer solution (8 M guanidine hydrochloride, 10 mM sodium dihydrogen phosphate, 20 mM NaCl, 1 mM Tris-HCl, pH 7.0) so that a concentration became 100 mg/mL, and dissolved by stirring with a stirrer at 60° C. for 30 minutes. After dissolution, dialysis was carried out with water using a dialysis tube (cellulose tube 36/32 manufactured by Sanko Junyaku Co., Ltd.). The white aggregated protein obtained after dialysis was recovered by centrifugation, the water content was removed with a freeze dryer, and the freeze-dried powder was recovered.

### (4) Preparation of Spinning Solution (Dope Solution)

The freeze-dried powder of the spider silk protein (PRT799) obtained in (3) was added to formic acid (manufactured by Asahi Chemical Co., Ltd.) so that a concentration became 26% by mass, and then dissolved at 40° C. for 1 hour. Thereafter, dust and bubbles were removed to obtain a dope solution 1.

Similarly, the freeze-dried powder of the spider silk protein (PRT966) obtained in (3) was added to formic acid (manufactured by Asahi Chemical Co., Ltd.) so that a concentration became 26% by mass, and then dissolved at 70° C. for 1 hour. Thereafter, dust and bubbles were removed to obtain a dope solution 2.

### (5) Evaluation of Fiber-Forming Ability

A 10 mL syringe was filled with the dope solution 1 or the dope solution 2 obtained in (4), and using a table-top spinning apparatus, the solution was discharged from a nozzle having a nozzle diameter of 0.2 jam into a coagulation liquid for spinning, and a fiber-forming ability was visually evaluated. An extrusion speed was 0.075 mL/min. The coagulated raw yarn was wound at a linear speed of 2.39 m/min. The types of coagulation liquid used are as shown in Table 6.

TABLE 6

| | | | Spider | Fiber- |
|---|---|---|---|---|
| | | Dope | silk | forming |
| | Coagulation liquid | solution | protein | ability |
| Test | 2-Propanol | Formic acid | PRT966 | C |
| Example 1 | | | | |
| Test | Methanol | | | B |
| Example 2 | | | | |
| Test | Acetone | | | A |
| Example 3 | | | | |
| Test | Acetone:water = | | | B |
| Example 4 | 7:3 (mass ratio) | | | |
| Test | Acetone:water = | | | C |
| Example 5 | 5:5 (mass ratio) | | | |
| Test | Acetone:formic | | | A |
| Example 6 | acid = 8:2 | | | |
| | (mass ratio) | | | |
| Test | Acetone:formic | | | B |
| Example 7 | acid = 7:3 | | | |
| | (mass ratio) | | | |
| Test | Acetone:formic | | | B |
| Example 8 | acid = 6:4 | | | |
| | (mass ratio) | | | |
| Test | Acetone:formic | | | C |
| Example 9 | acid = 5:5 | | | |
| | (mass ratio) | | | |
| Test | Methanol | Formic acid | PRT799 | A |
| Example 10 | | | | |
| Test | Acetone | | | A |
| Example 11 | | | | |
| Test | Acetone:water = | | | B |
| Example 12 | 7:3 (mass ratio) | | | |
| Test | Acetone:water = | | | C |
| Example 13 | 5:5 (mass ratio) | | | |

The evaluation results of the fiber-forming ability are collectively shown in Table 6. The evaluation standard for the fiber-forming ability are as shown below.

A (VG): Fibers were formed. The obtained fibers were flexible and uniform.

B (G): Fibers were formed. The obtained fibers were flexible.

C (S): Fibers were formed.

D (P): Fibers were not formed.

By using ketone (acetone) as the coagulation liquid, an extremely favorable fiber-forming ability was exhibited in both cases of PRT966 (hydrophobic protein) and PRT799 (hydrophilic protein) (Test Example 3 and Test Example 11). In addition, a favorable fiber-forming ability was also exhibited by using the coagulation liquid obtained by mixing acetone with water or formic acid (Test Examples 4 to 9, Test Example 12, and Test Example 13).

### (6) Evaluation of Stress

Using the dope solution 2, protein fibers were spun under the same conditions as those of (5). In addition, protein fibers having different draw ratios were spun while changing a winding speed (linear speed). Monofilaments of the spun protein fiber were attached to a square-shaped (Hollow square) mount one by one, and a diameter of the fiber was measured with a microscope from the side surface. Next, the mount itself was set to a tensile tester (tensile tester 3342, manufactured by Instron Co., Ltd.), the side portion of the mount was cut off, and then measurement was started. Measurement conditions were as follows: a tensile speed of 10 cm/min, a load cell capacity of 10 N, and a gripping jig distance of 20 mm.

A stress was calculated from a measured test force and an area calculated from the diameter of the fiber. The stress was calculated as a relative value with the stress in Test Example 3-1 as 100%. The results are collectively shown in Table 7.

TABLE 7

| Test | Test | Test | Test | Test | Test | Test | Test |
|---|---|---|---|---|---|---|---|
| Example | Example | Example | Example | Example | Example | Example | Example |
| 3-1 | 3-2 | 3-3 | 3-4 | 3-5 | 3-6 | 3-7 | 3-8 |

| | | | | | | |
|---|---|---|---|---|---|---|
| Coagulation | Acetone | % By mass | 100 | 80 | 70 | 60 |
| liquid | Formic acid | % By mass | 0 | 20 | 30 | 40 |

| | | | | | | | | | |
|---|---|---|---|---|---|---|---|---|---|
| Draw ratio | Times | 4.0 | 5.8 | 4.0 | 5.5 | 4.0 | 5.0 | 4.0 | 4.2 |
| Stress | (%) | 100 | 131 | 110 | 146 | 133 | 144 | 134 | 168 |

When the coagulation liquid prepared by mixing formic acid with acetone was used, the stress of the obtained protein fiber tended to be improved.

### (7) Evaluation of BD Maximum Draw Ratio

A 10 mL syringe was filled with the dope solution 1 or the dope solution 2, and using a table-top spinning apparatus, the solution was discharged from a nozzle having a nozzle diameter of 0.2 μm into a coagulation liquid. An extrusion speed was 0.075 mL/min. The coagulated raw yarn was wound around a winder. The types of coagulation liquid used are as shown in Table 8. A rotation speed of the winder was increased, and the rotation speed at the time when the fiber was fractured was read to obtain the BD maximum draw ratio. The BD (Bath Draft) is a ratio of the rotation speed of a first roller in a first coagulation bath to a discharge linear speed of the undiluted solution extruded from the nozzle, and the BD maximum draw ratio is the maximum draw ratio drawable between the nozzle and the first roller. The results are shown in Table 8. In Table 8, “Ratio” is a relative value of the BD maximum draw ratio when the BD maximum draw ratio of Test Example 4-2 (coagulation liquid: methanol) is set to 1 for Test Examples 4-1 to 4-3, and is a relative value of the BD maximum draw ratio when the BD maximum draw ratio of Test Example 4-4 (coagulation liquid: methanol) is set to 1 for Test Examples 4-4 and 4-5.

TABLE 8

| | Coagulation | | Spider | |
|---|---|---|---|---|
| | liquid | Dope solution | silk protein | Ratio |
| Test | 2-Propanol | Formic acid | PRT966 | 0.28 |
| Example 4-1 | | | | |
| Test | Methanol | | | 1.00 |
| Example 4-2 | | | | |
| Test | Acetone | | | 1.35 |
| Example 4-3 | | | | |
| Test | Methanol | Formic acid | PRT799 | 1.00 |
| Example 4-4 | | | | |
| Test | Acetone | | | 1.27 |
| Example 4-5 | | | | |

When acetone was used as the coagulation liquid, the BD maximum draw ratio was the highest (Test Example 4-3 and Test Example 4-5).

### (8) Manufacture Example 1

The freeze-dried powder of the spider silk protein (PRT799) obtained in (3) was added to formic acid (manufactured by Asahi Chemical Co., Ltd.) so that a concentration became 28% by mass, and then dissolved at 40° C. for 1 hour while stirring. Then, dust and bubbles were removed to obtain a spinning raw liquid (dope solution A). A viscosity of the dope solution A was 15,000 to 20,000 cP.

Similarly, the freeze-dried powder of the spider silk protein (PRT966) obtained in (3) was added to formic acid (manufactured by Asahi Chemical Co., Ltd.) so that a concentration became 24% by mass, and then dissolved at 70° C. for 1 hour while stirring. Then, dust and bubbles were removed to obtain a spinning raw liquid (dope solution B). A viscosity of the dope solution B was 10,000 to 15,000 cP.

Using the spinning raw liquid obtained under the above conditions, a spun and drawn protein fiber was manufactured by dry-wet-type spinning using a spinning apparatus corresponding to the spinning apparatus shown in FIG. 4. The spinning apparatus used was the same as the spinning apparatus shown in FIG. 4 except that it included a second coagulation bath and a third coagulation bath between the first coagulation bath and the wet heat drawing apparatus (washing bath). Methanol or acetone was used as a coagulation liquid. In addition, water was used as a washing solution in the washing bath. Other conditions are as follows.

Nozzle diameter of discharge part: 0.1 mm

Discharge rate: 3 cc/min

The maximum draw ratio was evaluated by the spinning apparatus shown in FIG. 4. The evaluated point was drawing by the washing bath. As an evaluation method, a draw ratio at which the fiber was fractured was taken as the maximum draw ratio while increasing the draw ratio by 1.

The results are shown in Table 9.

TABLE 9

| | | Coagulation liquid | |
|---|---|---|---|
| Spider silk protein | | Methanol | Acetone |
| PRT799 | Maximum | 5.5 to 6 times | 6 to 6.5 times |
| | draw ratio | | |
| | of drawing | | |
| | by washing | | |
| | bath | | |
| PRT966 | Maximum | 3 to 3.5 times | 4 to 4.5 times |
| | draw ratio | | |
| | of drawing | | |
| | by washing | | |
| | bath | | |

When acetone was used as the coagulation liquid, the maximum draw ratio in the washing bath was higher than the case in which methanol was used. Based on this result, it is expected that a stress of the fiber formed by using acetone will be improved.

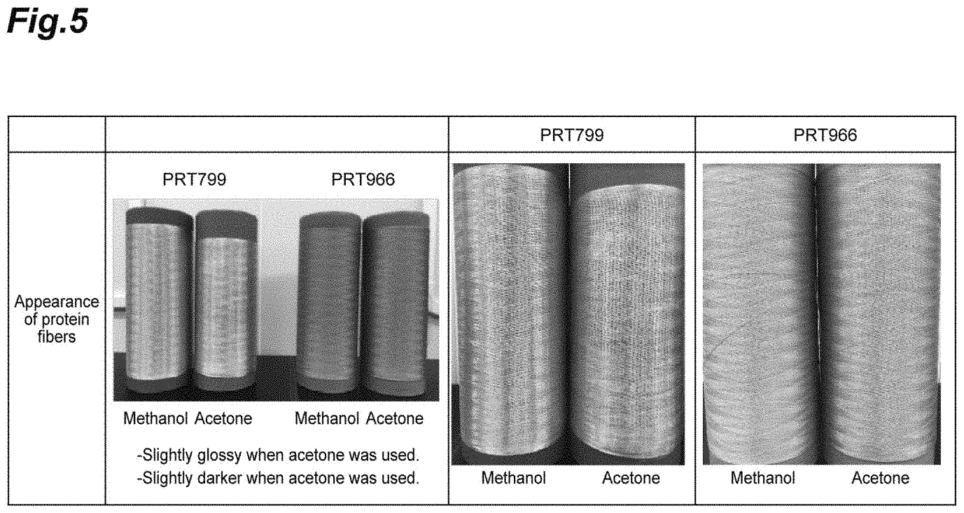

FIG. 5 shows photographs showing an appearance of protein fibers manufactured in Manufacture Example 1. When acetone was used as the coagulation liquid, a more glossy protein fiber was obtained as compared with the case in which methanol was used.

### (9) Manufacture Example 2

Using the spinning raw liquid (dope solution B) prepared in Manufacture Example 1, a protein fiber was produced in the same manner as in Manufacture Example 1. The gloss of the obtained protein fiber was visually determined. The evaluation standard for the gloss are as follows.

A (VS): Very strong

B (S): Strong

C (W): Weak

D (N): No gloss.

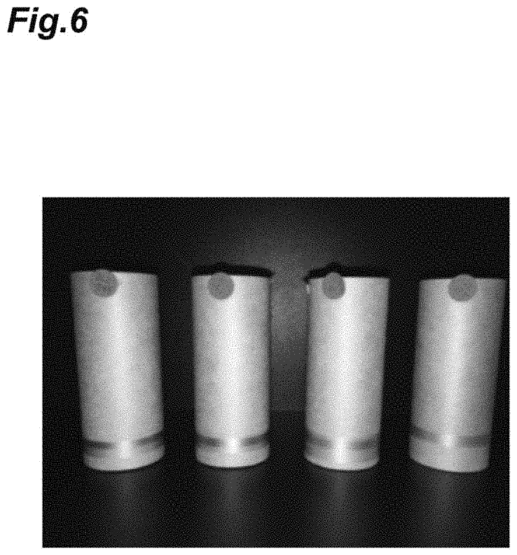

The evaluation results are shown in Table 10 and FIG. 6. Table 10 collectively shows the results of evaluating the gloss of the protein fibers (spider silk protein: PRT966, dope solvent: methanol) manufactured in Manufacture Example 1.

TABLE 10

| | Coagulation liquid | Dope solution | Fibroin | Gloss |
|---|---|---|---|---|
| Manufacture | Acetone | Formic acid | PRT966 | A |
| Example 2-1 | | | | |
| Manufacture | Acetone:formic acid = | | | A |
| Example 2-2 | 8:2 (mass ratio) | | | |
| Manufacture | Acetone:formic acid = | | | A |
| Example 2-3 | 7:3 (mass ratio) | | | |
| Manufacture | Acetone:formic acid = | | | B |
| Example 2-4 | 6:4 (mass ratio) | | | |
| Manufacture | Methanol | Formic acid | PRT966 | B |
| Example 1 | | | | |

FIG. 6 shows photographs showing an appearance of protein fibers manufactured in Manufacture Example 2. The protein fibers of Manufacture Examples 2-1 to 2-4 were disposed in order from the left. As compared with the case in which methanol was used as the coagulation liquid, there was a strong gloss even when the coagulation liquid in which formic acid was added to acetone was used as in the case of using 100% acetone as the coagulation liquid.

### REFERENCE SIGNS LIST

1 Extrusion apparatus
2 Undrawn yarn production apparatus
3 Wet heat drawing apparatus
4 Drying apparatus
6 Spinning raw liquid
10 Spinning apparatus
20 Coagulation bath
21 Washing bath
36 Protein fiber

### SEQUENCE LISTING

1. A method for manufacturing a protein fiber, the method comprising:
a step of bringing a spinning raw liquid containing a spider silk protein and a solvent into contact with a coagulation liquid to coagulate the spider silk protein,
wherein the coagulation liquid contains a ketone.

2. The manufacturing method according to claim 1, wherein the ketone is a water-soluble ketone.

3. The manufacturing method according to claim 1, wherein the ketone is acetone.

4. The manufacturing method according to claim 1, wherein a content of the ketone in the coagulation liquid is 60% by mass or more with respect to a total amount of the coagulation liquid.

5. The manufacturing method according to claim 1, wherein the coagulation liquid further contains at least one selected from the group consisting of the solvent, a dissolution promoter, and water.

6. The manufacturing method according to claim 1, wherein the solvent contains formic acid.

7. The manufacturing method according to claim 1,
wherein the coagulation liquid further contains formic acid, and
a content of the formic acid is 40% by mass or less with a total content of the ketone and the formic acid being 100% by mass.

8. The manufacturing method according to claim 1, wherein the spinning raw liquid further contains a dissolution promoter.

9. The manufacturing method according to claim 1, wherein the spider silk protein is a hydrophilic spider silk protein.

10. The manufacturing method according to claim 9, wherein a content of the ketone in the coagulation liquid is 70% by mass or more with respect to a total amount of the coagulation liquid.

11. The manufacturing method according to claim 1, further comprising a step of drawing the coagulated spider silk protein.
